(19) United States
(12) Patent Application Publication
(10) Pub. No.: US 2021/0388393 A1
(43) Pub. Date: Dec. 16, 2021

(54) PLGA-PEG/PEI NANOPARTICLES AND METHODS OF USE

(71) Applicant: ANN & ROBERT H. LURIE CHILDREN'S HOSPITAL OF CHICAGO, CHICAGO, IL (US)

(72) Inventor: Youyang Zhao, Deerfield, IL (US)

(21) Appl. No.: 17/284,262

(22) PCT Filed: Oct. 11, 2019
(86) PCT No.: PCT/US19/55787

§ 371 Date: Apr. 9, 2021

Related U.S. Application Data
(60) Provisional application No. 62/744,949, filed on Oct. 12, 2018.

(51) Int. Cl. C12N 15/90; A61K 9/51; C12N 15/11; C12N 9/22; C12N 15/88

(52) U.S. Cl. CPC C12N 15/907; A61K 9/5153; C12N 15/11; C12N 2800/80; C12N 15/88; C12N 2310/20; C12N 9/22

(57) ABSTRACT

Provided herein is a composition comprising a nanoparticle comprising poly(lactic acid-co-glycolic acid) (PLGA)-b-polyethylene glycol (PEG) (PLGA-PEG) copolymer formulated with polyethylenimine (PEI), and one or more cargo molecules (e.g., a nucleic acid molecule with or without a small molecule compound) associated with the nanoparticle. The nucleic acid molecule may be a plasmid or minicircle DNA expressing a gene or genes, CRISPR/Cas9 components, or an RNA molecule (e.g., small interfering RNA, miRNA, or lncRNA). Also provided are methods for delivering a cargo molecule to a cell in vitro and in vivo using the aforementioned composition.

### CROSS-REFERENCE TO RELATED APPLICATIONS

This application is a national phase application under 35 U.S.C. § 371 of PCT International Application No. PCT/US2019/055787, filed Oct. 11, 2019, which claims the benefit of U.S. Provisional Patent Application No. 62/744,949 filed Oct. 12, 2018, which is incorporated by reference herein.

### STATEMENT REGARDING FEDERALLY SPONSORED RESEARCH OR DEVELOPMENT

This invention was made with government support under HL123957, HL125350, HL133951, and HL077806 awarded by the National Institutes of Health. The government has certain rights in the invention.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 10,139 Byte ASCII (Text) file named “36917-252_ST25.txt,” created on Apr. 9, 2021.

### FIELD

The disclosure is directed nanoparticles comprised of a poly(lactic acid-co-glycolic acid) (PLGA)-b-polyethylene glycol (PEG) copolymer (PLGA-b-PEG, or PLGA-PEG) formulated with polyethylenimine (PEI) for the delivery of nucleic acid molecules.

### BACKGROUND OF THE INVENTION

The efficient delivery of nucleic acids, such as plasmid DNA and its derivatives (e.g., minicircle DNA) and RNA (e.g., small interfering RNA (siRNA), antisense RNA, microRNA (miRNA), and long noncoding RNA (lncRNA)) to cultured cells or to live animals and humans is critically important for biomedical research and the development of therapeutic agents for various diseases.

Despite the efficacy of viral vectors for gene delivery, viral vectors may induce immune responses and other severe side effects (Raper et al., Mol. Genet. Metab., 80: 148-158 (2003); Manno et al., Nat. Med., 12: 342-347 (2006); and Howe, J. Clin. Invest., 118: 3143-3150 (2008)), which limit their clinical utility. For example, adeno-associated virus-mediated delivery of the CRISPR/Cas9 system permits rapid genome editing in animals, (AAV) (Cox et al., Nat Med., 21: 121-131 (2015); Yin et al., Nat Biotechnol, 35: 1179-1187 (2017); Nelson et al., Science, 351: 403-407 (2016); and Mali et al., Science, 339: 823-826 (2013)), but is problematic for several reasons. First, the AAV vector is highly immunogenic and has a low packaging capacity (restricted to 4.7 kb in AAVs) (Nelson et al., supra; and Carroll et al., Proc. Natl. Acad. Sci. USA, 113: 338-343 (2016)). Second, extended expression of Cas9 from AAV may cause unwanted DNA damage. Third, AAV-mediated gene expression is limited to the liver after systemic administration.

To circumvent the disadvantages associated with viral vector delivery systems, non-viral gene delivery methods and reagents have been explored, including liposomes, polycationic polymers, and organic or inorganic nanoparticles. While some of these systems exhibit improved safety profiles, many are limited by low gene transfer efficiency both in vitro and in vivo and primarily accumulate in the liver. In addition, many non-viral delivery systems are not capable of cell-specific gene delivery, which is especially needed for treating various types of diseases including cardiovascular diseases and cancer. Thus, there remains a need for compositions and methods that efficiently deliver genes to cells in vitro and in vivo with limited associated toxicity.

### BRIEF SUMMARY OF THE INVENTION

Provided herein is a composition comprising: (a) a nanoparticle comprising poly(lactic acid-co-glycolic acid) (PLGA)-b-polyethylene glycol (PEG) copolymer (PLGA-b-PEG or PLGA-PEG) formulated with polyethylenimine (PEI), and (b) one or more cargo molecules associated with the nanoparticle. Also provided herein is a method of delivering one or more cargo molecules to a cell, which comprises contacting the cell with the aforementioned composition.

### BRIEF DESCRIPTION OF THE DRAWINGS

FIG. 1 is a schematic diagram which illustrates the preparation of PEI-coated PLGA-PEG nanoparticles using an oil-in-water double emulsion method.

FIG. 2A and FIG. 2B are graphs which illustrate the size distribution of PLGA-PEG (FIG. 2A) nanoparticles and PEI-coated PLGA-PEG nanoparticles (FIG. 2B). PLGA55,000-PEG5,000 nanoparticles were mixed with PEI 25,000 (molecular weight) at a weight ratio of 1:4 and incubated for 72 hours at room temperature. Following centrifugation of 19,000 g for 20 minutes, the supernatants were collected for determination of size distribution.

FIG. 3A and FIG. 3B are graphs which illustrate Zeta potential of PLGA-PEG nanoparticles (FIG. 3A) and PEI 25,000-coated PLGA-PEG nanoparticles (FIG. 3B).

FIG. 4A is a schematic diagram illustrating the all-in-one CRISPR^{CAG} plasmid DNA expressing Cas9 under the control of CAG promoter and gRNA driven by U6 promoter. FIG. 4B is a graph which illustrates size distribution of PLGA-PEG/PEI 25,000/plasmid DNA nanoparticles. Plasmid DNA was mixed with PLGA-PEG/PEI 25,000 nanoparticles at the ratio of 1 μg DNA and 3 μl of the PLGA-PEG/PEI 25,000 nanoparticles for 30 minutes. FIG. 4C is an image of a DNA electrophoresis gel demonstrating that PLGA-PEG/PEI 25,000 nanoparticles neutralized the negative charge of CRISPR plasmid DNA and blocked its movement in the gel. The lanes of the gel are labeled as follows: M, molecular weight. 0, CRISPR plasmid DNA without nanoparticles. 1-6, CRISPR plasmid DNA mixed with various volumes of PLGA-PEG/PEI nanoparticles.

FIG. 5A is a series of fluorescent microscopy images which illustrate the optimization of the ratio of nanoparticles:plasmid DNA for highly efficient transfection in vitro. Various volumes of nanoparticles were mixed with 1 μg of CRISPR plasmid DNA and added to Hepa-1c1c7 cells for 48 hours. Expression of GFP was detected using fluorescent microscopy. The ratio of 4 μl PP/PEI:1 μg DNA was selected for transfection experiments. FIG. 4B is a series of micrographs showing more than 90% Hepa-1c1c7 cells expressing GFP at 48 h post-nanoparticle-mediated transfection of CRISPR plasmid DNA (right panel). The left panel shows phase-contrast microscopy of all cells. Scale bar, 100 μm.

FIG. 6 is a series of fluorescent microscopy images which illustrate the efficiency of PP/PEI nanoparticle-mediated transfection (2 μl and 4 μl) in the mouse Neuro-2A neuroblastoma cell line and primary cultures of mouse lung fibroblasts.

FIG. 7A is a graph which illustrates Sanger sequencing decomposition analysis using TIDE software. 80% genome editing efficiency was observed with CRISPR plasmid DNA expressing gRNA1. FIG. 7B is a schematic diagram of the strategy for quantitative PCR identification of potent gRNAs. The forward primer containing deleted nucleotides (e.g., TG) will only amplify the wild type genomic DNA (gDNA) or cDNA. FIG. 7C is a graph illustrating quantitative PCR analysis of genomic DNA, which shows that highly efficient genome editing was induced by gRNA1 targeting Pik3cg. FIG. 7D is a graph illustrating quantitative PCR analysis of cDNA converted from total RNA, which shows a marked decrease in WT Pik3cg mRNA expression in gRNA1-transfected cells. Efficiency of genome editing demonstrated by these PCR-based analyses was similar to that identified by Sanger sequencing decomposition analysis. Bars represent means.

FIG. 8 illustrates the biodistribution of PLGA-PEG-based nanoparticles in adult mice. FIG. 8A is a schematic diagram of the preparation of fluorescent dye (Coumarin-6)-encapsulated PP nanoparticles. FIG. 8B is a micrograph of fluorescent tomography by IVIS imaging of live mice demonstrating that the PLGA-PEG nanoparticles were distributed throughout the whole body without specific accumulation in the liver and other organs. The image was taken 5 hours after injection of coumarin 6-loaded PLGA-PEG nanoparticles (no plasmid DNA) via the retro-orbital venous plexus. FIG. 8C is a schematic diagram illustrating the procedures to generate PLGA-PEG/PEI nanoparticles for plasmid DNA delivery. PEG-b-PLGA was used for generation of the PLGA-PEG (PP) nanoparticles and then incubated with PEI to generate PP/PEI nanoparticles which were incubated with plasmid DNA for intravenous delivery to mice. FIG. 8D is a graph which illustrates a quantitative PCR analysis showing plasmid DNA distribution in various organs at 8 hours post-iv PLGA-PEG/PEI/DNA administration (sk muscle=skeletal muscle).

FIG. 9A is a schematic diagram which illustrates nanoparticle-mediated delivery of the CRISPR system to adult mice. FIG. 9B is a graph which illustrates a quantitative PCR analysis showing a marked decrease of wild-type Pik3cg genomic DNA (gDNA) in freshly isolated lung ECs (CD31⁺) in mice treated with CRISPR^{CDH5}/gRNA1 plasmid DNA-loaded PP/PEI nanoparticles but not in mice treated with either plasmid DNA-loaded PLGA nanoparticles which have no PEG block or plasmid DNA-loaded PEI mixtures with the same amount (PEI 1×) or 3 times of PEI (PEI×3) as used in PP/PEI nanoparticles. 7 days post-administration, lung tissues were collected for cell isolation by magnetic beads. Genomic DNA was extracted from these freshly isolated cells followed by quantitative PCR analysis. FIG. 9C is a graph which illustrates a quantitative PCR analysis showing a marked decrease of wild-type Pik3cg genomic DNA (gDNA) in freshly isolated lung ECs (CD31⁺) but not in non-ECs (CD31⁻) in mice treated with CRISPR^{CDH5}/gRNA1 plasmid DNA-loaded nanoparticles. gRNA3 failed to reduce wild-type Pik3cg genomic DNA. “CTL” indicates control mice treated with PP/PEI nanoparticles but no plasmid DNA. Lungs were collected and ECs were isolated with anti-CD31 magnetic beads. FIG. 9D is a graph which illustrates Sanger sequencing decomposition analysis showing 40% efficient genomic editing in CRISPR^{CDH5}/gRNA1 nanoparticle-treated mice with selectivity for ECs isolated from lungs. FIG. 9E is an image of a Western blot demonstrating diminished p110γ protein expression in ECs but not in non-ECs isolated from lungs of CRISPR^{CDH5}/gRNA1 nanoparticle-treated mice as compared to gRNA3-treated mice. p110γ protein expression, although at lower levels, was similar in non-ECs. Expression of p110α isoform of PI3K was not affected by gRNA1 nanoparticle treatment. FIG. 9F includes graphs which illustrate the quantification of protein expression based on Western blot band intensity. FIG. 9G is a series of micrographs of immunofluorescent staining showing diminished p110γ expression in pulmonary vascular ECs obtained from CRISPR^{CDH5}/gRNA1 nanoparticle-treated mice. Cryosections were immunostained with anti-CD31 (marker for ECs) and anti-p110γ. Nuclei were counterstained with DAPI. Scale bar, 50 μm. Bars represent means. ***P<0.001 student's t test in FIGS. 9B and D and two-way ANOVA with Bonferroni post-hoc analysis in FIGS. 9C and 9D.

FIG. 10A is a graph depicting measurement of pulmonary transvascular EBA flux demonstrating defective vascular repair in gRNA1 nanoparticle-treated mice at 72 hours post-LPS challenge as compared to gRNA3 nanoparticle-treated mice. “Basal” indicates gRNA1 nanoparticle-treated mice without LPS challenge. FIG. 10B is a graph depicting persistently elevated lung MPO activity in gRNA1 nanoparticle-treated mice at 72 hours post-LPS challenge. FIG. 10C is a graph depicting a marked increase in expression of proinflammatory genes in lungs of gRNA1 nanoparticle-treated mice at 72 hours post-LPS challenge. FIGS. 10D and 10E are graphs which illustrate results of quantitative RT-PCR analysis showing inhibited expression of the transcription factor FoxM1 (FIG. 10D) and its target genes (FIG. 10E) at 72 hours post-LPS challenged in lungs of gRNA1 nanoparticle-treated mice, as compared to gRNA3-treated mice. Bars represent means. **P<0.01, ***P<0.001 using two-way ANOVA with Bonferroni post-hoc analysis in FIG. 10A-10E.

FIG. 11A and FIG. 11B are graphs which illustrate the results of quantitative PCR analysis demonstrating efficient genome editing of wild-type Pik3cg gDNA (FIG. 11A) and cDNA (FIG. 11B) in cardiovascular ECs but not in non-ECs of Pik3cg gRNA1 nanoparticle-treated mice. Hearts were collected, and ECs were isolated with anti-CD31 magnetic beads. FIG. 11C is a graph which illustrates the results of Sanger sequencing decomposition analysis showing that indels occurred exclusively in cardiovascular ECs at a rate of 40%. FIG. 11D is an image of a Western blot showing diminished expression of p110γ in cardiovascular ECs of Pik3cg gRNA1 nanoparticle-treated mice. p110γ expression was similar in non-ECs from gRNA1 nanoparticle-treated mice as compared to gRNA3 nanoparticle-treated mice. FIG. 11E is a series of immunofluorescent micrographs showing diminished p110γ expression in cardiovascular ECs of gRNA1 nanoparticle-treated mice. Cryosections of mouse hearts were immunostained with anti-p110γ and anti-CD31. Nuclei were counterstained with DAPI (blue). Scale bar, 50 μm. Bars represent means. *** P<0.001 using two-way ANOVA with Bonferroni post-hoc analysis in FIGS. 11A and 11B; unpaired two-tailed student's t test was used in FIG. 11C.

FIG. 12A and FIG. 12.B are graphs illustrating the results of quantitative PCR analysis demonstrating efficient genome editing wild-type Pik3cg gDNA (FIG. 12A) and cDNA (FIG. 12B) with selectivity for ECs of the abdominal aorta isolated from gRNA1 nanoparticle-treated mice. FIG. 12C is a graph which illustrates the results of Sanger sequencing decomposition analysis showing greater than 40% indels occurring in aortic ECs but not in non-ECs. FIG. 12D is a series of immunofluorescent micrographs showing that p110γ expression was markedly decreased in aortic ECs obtained from gRNA1 nanoparticle-treated mice as compared to gRNA3-treated mice. Scale bar, 50 μm. Bars represent means. *** P<0.001 using two-way ANOVA with Bonferroni post-hoc analysis in FIGS. 12A and 12B; unpaired two-tailed student's t test was used in FIG. 12C.

FIG. 13A is a graph illustrating the results of Sanger sequencing decomposition analysis showing 35% indels occurring in ECs freshly isolated from the peripheral blood vessels in mouse thigh muscle but not in non-ECs. FIG. 13B is a series of immunofluorescent micrographs showing that p110γ expression was markedly decreased in peripheral vascular ECs obtained from gRNA1 nanoparticle-treated mice in contrast to gRNA3-treated mice. Vascular ECs were immunostained with anti-CD31, while p110γPI3K was immunostained with anti-p110γPI3K. Nuclei were counterstained with DAPI. Scale bar, 50 μm.

FIG. 14A is a graph which illustrates the results of quantitative PCR analysis showing a 38% decrease of wild type (WT) genomic DNA in lung tissues of Vegfr2 gRNA3-transduced mice. FIG. 14B is a graph which illustrates the results of quantitative PCR analysis showing a 55% reduction of wild type Vegfr2 cDNA in lung tissues of gRNA3-transduced mice, indicating highly efficient genomic editing in vivo. FIG. 14C is a series of fluorescent micrographs demonstrating diminished expression of VEGFR2 in pulmonary vascular ECs of gRNA3-treated mice (n=4 mice/group; Scale bar, 50 μm).

FIG. 15 is a series of immunofluorescent micrographs showing diminished expression of VEGFR2 in cardiovascular (heart) ECs of Vegfr2 gRNA3-transduced mice as compared to scramble sequence-transduced mice. Scale bar, 50 μm.

FIG. 16 is a series of immunofluorescent micrographs showing that Vegfr2 gRNA induced highly efficient genome editing in aortic vascular ECs in adult Vegfr2 gRNA3-transduced mice compared to scramble sequence-transduced mice. Scale bar, 50 μm.

FIG. 17 is a graph showing that modified PEI25K by succinylation (suPEI25k) also induced highly efficient genome editing in lung ECs in adult Pik3cg gRNA1-transduced mice compared to CTL mice. **, P<0.01. (n=3 mice).

FIG. 18 is a graph which illustrates that novel formulation of PP nanoparticles with low molecular weight PEI, e.g., PEI600 Da also induced highly efficient genome editing in lung ECs in adult Pik3cg gRNA1-transduced mice compared to CTL mice.

FIG. 19 is a graph that demonstrates marked genome editing effects of PP/cPEI nanoparticles formulated with crosslinked low molecular weight PEIs (cPEI600 Da and cPEI1200 Da) by disulfide linkers in lung ECs from Pik3cg gRNA1-transduced mice compared to CTL mice. * P<0.05, **, P<0.01. n=3/group.

FIG. 20 is a series of micrographs of Cxcr4 immunostaining demonstrating restored Cxcr4 expression in pulmonary vascular ECs in Cxcr4^{ΔEC} (CKO) mice administered with plasmid DNA expressing Cxcr4 under the control of human CDH5 promoter (Cxcr4). Lung sections were collected at 48 hours post-nanoparticle:plasmid DNA administration (i.e., 60 hours post-LPS) for immunostaining with endothelial cell marker anti-CD31 (green) and Cxcr4 (red), respectively. Nuclei were counterstained with DAPI (blue). V, vessel. Scale bar, 20 μm.

FIG. 21 is a graph which illustrates the results of quantitative RT-PCR analysis showing persistent gene expression after nanoparticle-mediated gene delivery. 20 μg of plasmid DNA expressing KLF4 under the control of the CDH5 promoter were mixed with the nanoparticles and administered to adult mice. At the indicated times, lung tissues were collected for EC and non-EC isolation. KLF4 expression was quantified by quantitative RT-PCR analysis. “CTL” indicates control mice treated with nanoparticle only without plasmid DNA.

### DETAILED DESCRIPTION OF THE INVENTION

The present disclosure is predicated, at least in part, on the discovery that polyethylenimine (PEI)-formulated nanoparticles composed of poly(lactic acid-co-glycolic acid) (PLGA)-b-polyethylene glycol (PEG) (PLGA-PEG) co-polymer are capable of delivering nucleic acids into cultured cells with an efficiency similar to or greater than more widely used transfection reagents (e.g., lipofectamine) and uniquely into live animals with high efficiency. These nanoparticles are distributed throughout the entire organism, instead of concentrating in the liver as observed for other nanoparticles and recombinant viral vectors. It will be appreciated that nanoparticles can be engineered to harness optimal targeting of drugs to specific cells and tissues and to optimize drug-loading capacity, allowing for improved pharmacokinetics, safe and effective drug delivery, and enhanced bioavailability of therapeutics (Ulbrich et al., J. R. Soc. Interface, 7 (Suppl. 1): S55-S66 (2010); and Prosperi et al., Semin. Immunol., 34, 61-67 (2017)).

The term “nanoparticle,” as used herein, refers to a particle having at least one dimension in the range of about 1 nm to about 1000 nm, including any integer value between 1 nm and 1000 nm (including about 1, 2, 5, 10, 20, 50, 60, 70, 80, 90, 100, 200, 500, 1000 nm, and all integers and fractional integers in between). In some embodiments, the nanoparticle has at least one dimension, e.g., a diameter, of about 100 nm. In other embodiments, the nanoparticle has a diameter of about 200 nm, a diameter of about 500 nm, or a diameter of about 1000 nm (1 μm). Nanoparticles having a diameter of at least 1000 nm also may be referred to as a “microparticle.” Thus, the term “microparticle” includes particles having at least one dimension in the range of about one micrometer (μm), i.e., 1×10⁻⁶ meters, to about 1000 μm. The term “particle” as used herein is meant to include nanoparticles and microparticles.

Nanoparticles suitable for use in the presently disclosed compositions and methods may exist in a variety of shapes, including, but not limited to, spheroids, rods, disks, pyramids, cubes, cylinders, nanohelixes, nanosprings, nanorings, rod-shaped nanoparticles, arrow-shaped nanoparticles, teardrop-shaped nanoparticles, tetrapod-shaped nanoparticles, prism-shaped nanoparticles, and a plurality of other geometric and non-geometric shapes. In some embodiments, the disclosed nanoparticles have a spherical shape.

The nanoparticle may be of any composition that is suitable for efficient delivery of nucleic acids to cells. Several different types of nanoparticles have been developed that are suitable for nucleic acid delivery, including, for example, lipid-based nanoparticles (Pensado et al., Expert Opin Drug Deliv., 11: 1721-1731 (2014)), polymer-based nanoparticles (Gao et al., Acta Biomater, 25: 184-193 (2015)), and inorganic nanoparticles.

Lipid-based nanoparticles are composed of physiological lipids; hence, they are well tolerated, usually nontoxic, and are degraded to a nontoxic residue. Liposomes were one of the first developed lipid-based carriers characterized to be non-toxic, flexible, biocompatible, and completely biodegradable (see, e.g., Akbarzadeh et al., Nanoscale Res. Lett., 8: 102 (2013)). Liposomes are primarily composed of phospholipid bilayer vesicles containing phosphatidylcholine and phosphatidylethanolamine, the most common phospholipids found in nature, with other membrane bilayer constituents, such as cholesterol and hydrophilic polymers around each liposomal vesicle (Gregoriadis, G., Trends Biotechnol., 13: 527-537 (1995); and Chuang et al., Nanomaterials, 8: 42 (2018)). To enhance their circulation half-life and stability in vivo, liposomes may be conjugated with biocompatible polymers such as polyethylene glycol (PEG) (Torchilin, V. P., Nat Rev Drug Discov., 4: 145-160 (2005)). Liposomes can also be functionalized with targeting ligands to increase the accumulation of diagnostic and therapeutic agents within target cells.

Polymer-based nanoparticles typically are formed from biocompatible and biodegradable block co-polymers of different hydrophobicity (Chan et al., In: Cancer Nanotechnology; Grobmyer S R, Moudgil B M, editors. Vol. 624. Humana Press; 2010. pp. 163-175)). These copolymers spontaneously assemble into a core-shell micelle formation in an aqueous environment (Torchilin, V. P., Pharm Res., 24: 1-16 (2007)). Polymeric nanoparticles have been formulated to encapsulate hydrophilic and/or hydrophobic small drug molecules, as well proteins and nucleic acid macromolecules (Wang et al., Expert Opinion on Biological Therapy, 8: 1063-1070 (2008)). Several polymer-based nanoparticles, such as, for example, poly(lactic-co-glycolic acid) or poly(lactide-co-glycolide) (PLGA), polylactic acid or polylactide (PLA), poly glycolic acid or polyglycolide (PGA), polycaprolactone (PCL), poly (D,L-lactide) (PDLLA), chitosan, and PLGA-PEG have been developed for drug delivery and are in various stages of clinical trials (Devulapally R, Paulmurugan R., “Polymer nanoparticles for drug and small silencing RNA delivery to treat cancers of different phenotypes,” Wiley Interdisciplinary Reviews. Nanomedicine and Nanobiotechnology. 2014; 6(1): 10.1002/wnan.1242. doi:10.1002/wnan.1242).

Inorganic nanoparticles exhibit different capabilities depending on the chemical composition of their cores. A wide variety of inorganic nanoparticles are used in the art for biological applications. For example, semiconductor quantum dots are commercially available and offer an alternative to fluorescently labeled particles, while iron oxide nanoparticles have been approved for human use in magnetic resonance imaging (MRI) applications as contrast enhancers. Gold nanoparticles offer many size- and shape-dependent optical and chemical properties, biocompatibility, and facile surface modification (Wang E C, Wang A Z, Integrative Biology: Quantitative Biosciences from Nano to Macro, 6(1): 9-26 (2014)).

In some embodiments, the nanoparticle comprises poly(lactic acid-co-glycolic acid) (PLGA)-b-polyethylene glycol (PEG) (PLGA-PEG) co-polymer (referred to herein as “PLGA-PEG” nanoparticles). PLGA is a widely-used polymer in nanoparticles due to its biocompatibility, low toxicity, and well-documented utility for sustained drug release. PLGA has been approved by U.S. Food and Drug Administration and the European Medicine Agency for human use, and PLGA or PLGA-based nanoparticles have been widely employed for small molecule drug delivery applications (see, e.g., Dinarvand et al., International Journal of Nanomedicine, 6: 877-895 (2011); and Makadia H K, Siegel S J., Polymers, 3:1377-1397 (2011)). PLGA is a copolymer of polylactic acid and polyglycolic acid which can be synthesized in a wide range of molecular weights by ring-opening polymerization of cyclic dimers, i.e. lactide and glycolide, in the presence of metal catalysts (Nimesh, S., “Poly(D,L-lactide-co-glycolide)-based nanoparticles,” In: Gene Therapy: Potential Applications of Nanotechnology, Woodhead Publishing Series in Biomedicine, 2013, pp 309-329). Different forms of PLGA are known in the art, depending on the ratio of lactide to glycolide used for the copolymerization. PLGA breaks down into body metabolites, i.e. lactic and glycolic acid, by hydrolysis of ester bonds, which are removed by Kreb cycle. PLGA comprising any suitable ratio of lactide:glycolide (e.g., 50:50, 35:65, 25:75, 75:25, 65:35) may be employed for generation of the nanoparticle. (PLGA)-b-polyethylene glycol (PEG) (PLGA-PEG) co-polymer is a biocompatible, amphiphilic block copolymer composed of a hydrophilic PEG block and a hydrophobic PLGA block. These materials have been used in control release and nanoparticle formulation for drug encapsulation and delivery applications.

PLGA-PEG nanoparticles may be prepared using any suitable method known in the art for preparing polymer-based nanoparticles. Such methods include, but are not limited to, emulsion-solvent evaporation or diffusion, double emulsion, nanoprecipitation, salting out, dialysis, and supercritical fluid techniques (Rao J. P., Geckeler K. E., Progress in Polymer Science, 36: 887-913 (2011)). In some embodiments, a water-in-oil-in water (W/O/W) double emulsion solvent evaporation method may be used for encapsulation of hydrophilic agents, and a nanoprecipitation technique may be used for encapsulation of hydrophobic agents. In addition, PLGA-b-PEG raw material and PLGA-PEG nanoparticles are commercially available from a variety of sources and may be used in the context of the disclosure.

PLGA and PEG of any suitable molecular weight may be employed in the nanoparticle, and both are commercially available over a wide range of molecular weights. For example, the PLGA molecular weight may be 10,000 g/mol, 20,000 g/mol, 25,000 g/mol, 30,000 g/mol, 40,000 g/mol, 45,000 g/mol, 50,000 g/mol, 55,000 g/mol, 60,000 g/mol, 65,000 g/mol, 70,000 g/mol, 75,000 g/mol, 80,000 g/mol, 90,000 g/mol, 100,000 g/mol, or a range defined by any of the two foregoing values. The PEG molecular weight may be, for example, between about 300 g/mol to about 10,000,000 g/mol (e.g., about 600, 1,000, 5,000, 10,000 g/mol, or a range defined by any two of the foregoing values).

In some embodiments, the PLGA-PEG nanoparticle is coated or formulated with polyethylenimine (PEI). PEI is a synthetic cationic polymer with a repeating unit composed of an amine group and two carbon aliphatic CH₂CH₂ spacer. PEIs can compact DNA and RNA into complexes that are effectively taken up in cells, and therefore have been used in nucleic acid delivery and gene therapy applications (Boussif et al., Proc. Natl. Acad. Sci. U.S.A.; 92: 7297-7301 (1995); Godbey et al., Proc. Natl. Acad. Sci. U.S.A., 96: 5177-5181 (1999); Urban-Klein et al., Gene Ther., 12: 461-466 (2005); Xia et al., ACS Nano, 3(10): 3273-3286 (2009)). PEI also can be attached to nanoparticle surfaces through covalent and electrostatic interactions (Park et al., Int. J. Pharm., 359:280-287 (2008); Elbakry et al., Nano Lett., 9: 2059-2064 (2009); Fuller et al., Biomaterials, 29: 1526-1532 (2008); McBain et al., J. Mater. Chem., 17: 2561-2565 (2007); and Liong et al., Adv. Mater., 21: 1684-1689 (2009)). It will be appreciated that coating the PLGA-PEG nanoparticles with a polymer such as PEI may facilitate delivery of nucleic acids such as DNA and RNA and allow for nanoparticle targeting (such as, e.g., for in vivo genome editing applications or gene expression). PEI of any suitable molecular weight may be employed to coat the nanoparticle, and PEI is commercially available over a wide range of molecular weights (e.g., 400, 600, 800, 1200, 1800, 5000, 10000, and 25000 Da).

The PEI may be unmodified or modified (e.g., via succinylation or acetylation) to lower its potential toxicity or increase its nucleic acid condensation capacity. It will be appreciated that succinylation can lower the potential toxicity of large molecular weight PEI. (Zintchenko et al., Bioconjug Chem., 19: 1448-55 (2008)). Although large molecular weight PEIs (e.g., 25 kDa) are potentially toxic due to their aggregation and difficulty degrading, low molecular weight PEIs (e.g. Mw˜600 Da) are well tolerated but have low binding capacity for nucleic acids (Godbey et al., J Biomed Mater Res., 45: 268-275 (1999); Breunig et al., Proc Natl Acad Sci USA, 104: 14454-14459 (2007)). In some embodiments, the composition described herein comprises low molecular weight PEI formulated with the PLGA-PEG nanoparticle (e.g., 120:1 PEI600:PLGA-PEG), which increases nucleic acid binding capacity to levels similar to large molecular weight PEI. In other embodiments, the PEI polymer is the product of crosslinked low molecular weight PEI, e.g. crosslinked PEI600, PEI1200, and PEI1800, via various crosslinkers (e.g., disulfide, disimine, diacrylate).

The composition described herein comprises one or more cargo molecules associated with the nanoparticle. The terms “cargo” or “cargo molecule,” as used herein, refer to any entity (e.g. a small molecule, macromolecule or macromolecular complex), which may be delivered/transferred/is transferable across the membrane of a cell or into the cytosol or nucleus of a target cell. When two entities are “associated with” one another, as described herein, they are linked by a direct or indirect covalent or non-covalent interaction. In certain embodiments, the association is covalent. Ideally, the association is non-covalent. Suitable non-covalent interactions include, but are not limited to, hydrogen bonding, van der Waals interactions, hydrophobic interactions, magnetic interactions, electrostatic interactions, etc. In some embodiments, the association is electrostatic. In some embodiments, the one or more cargo molecules is one or more nucleic acid molecules. The terms “nucleic acid molecule,” “nucleic acid sequence,” and “polynucleotide” are synonymous and are intended to encompass a polymer of DNA or RNA, which can be single-stranded or double-stranded and which can contain non-natural or altered nucleotides. The terms include, as equivalents, analogs of either RNA or DNA made from nucleotide analogs and modified polynucleotides such as, though not limited to, methylated and/or capped polynucleotides. Nucleic acids are typically linked via phosphate bonds to form nucleic acid sequences or polynucleotides, though many other linkages are known in the art (e.g., phosphorothioates, boranophosphates, and the like).

The one or more nucleic acid molecules may be DNA, RNA, or combinations thereof (e.g., a DNA/RNA hybrid). In some embodiments, the nucleic acid molecule is a plasmid. The term “plasmid,” as used herein, refers to a small DNA molecule within a cell that is physically separated from a chromosomal DNA and can replicate independently (i.e., as an “episome”). Plasmids occur naturally in bacteria, archaea, and other eukaryotic organisms and commonly exist as small circular double-stranded DNA molecules. Synthetic plasmids are widely used in the art as vectors in molecular cloning, driving the replication of recombinant DNA sequences within host organisms. Plasmid DNA may be generated using routine molecular biology techniques, such as those described in, e.g., Green and Sambrook, Molecular Cloning: A Laboratory Manual (Fourth Edition), Cold Spring Harbor Laboratory Press (Jun. 15, 2012) or may be obtained from commercial sources.

In other embodiments, the one or more nucleic acid molecules may be a minicircle DNA. The term “minicircle DNA,” as used herein, refers to small excised, circular DNA fragments from a parental plasmid which is in generally free of bacterial plasmid DNA sequences. Minicircle DNA is used in the art as a vector for gene transfer into mammalian cells and has the advantage of reduced immunogenicity due to the lack of bacterial DNA sequences (Gaspar et al., Expert Opin Biol Ther 15(3):353-79 (2015)).

The plasmid or minicircle DNA may be any suitable recombinant plasmid that comprises a heterologous nucleic acid sequence to be delivered to a target cell, either in vitro or in vivo. The heterologous nucleic acid sequence may encode a gene product (e.g., a protein) of interest for the purposes of, for example, disease treatment or prevention, and may optionally be in the form of an expression cassette. The term “recombinant” refers to a polynucleotide of semisynthetic, or synthetic origin which either does not occur in nature or is linked to another polynucleotide in an arrangement not found in nature. The term “heterologous,” as used herein refers to a nucleic acid sequence obtained or derived from a genetically distinct entity from the rest of the entity to which it is being compared.

In some embodiments, the nucleic acid molecule associated with the nanoparticle is a DNA plasmid or minicircle DNA that comprises one or more nucleic acid sequences that express a gene (or genes) of interest to mediate genome editing or modification of a target gene or modulation of the expression levels of target gene(s). For example, the DNA plasmid or minicircle DNA may a gene, genome editor component(s), CRISPR/Cas9 components, or Cxrc4. In some embodiments, the DNA plasmid or minicircle DNA encodes and expresses components of the CRISPR/Cas9 gene editing system. CRISPR/Cas gene editing systems have been developed to enable targeted modifications to a specific gene of interest in eukaryotic cells. CRISPR/Cas gene editing systems are based on the RNA-guided Cas9 nuclease from the type II prokaryotic clustered regularly interspaced short palindromic repeats (CRISPR) adaptive immune system (see, e.g., Jinek et al., Science, 337: 816 (2012); Gasiunas et al., Proc. Natl. Acad. Sci. U.S.A., 109, E2579 (2012); Garneau et al., Nature, 468: 67 (2010); Deveau et al., Annu. Rev. Microbiol., 64: 475 (2010); Horvath and Barrangou, Science, 327: 167 (2010); Makarova et al., Nat. Rev. Microbiol., 9, 467 (2011); Bhaya et al., Annu. Rev. Genet., 45, 273 (2011); Cong et al., Science, 339: 819-823 (2013); and U.S. Pat. Nos. 8,697,359, 8,795,965, and 9,322,037). The CRISPR/Cas9 system is extensively used in the art to edit the genome of zygotes to generate various genetically modified animal species, including mice and rats. The use of CRISPR/Cas9 in postnatal or adult animals including canines and monkeys also is under investigation (Cong et al., Science 339, 819-823 (2013); Cox et al., supra, Doudna et al., Science, 346: 1258096 (2014); and Yin et al., supra). In addition to CRISPR/Cas9 systems, the nanoparticle composition described herein may be used to deliver other CRISPR/Cas systems known in the art, including, for example, CRISPR/Cas13, which induces RNA knockdown (Zetsche et al., Cell, 163, 759-771 (2015)) and CRISPR/Cpf129 (Kim et al., Nature Communications, 8 (14406): 14406 (2017)), and base editors (Gehrke et al., Nature Biotechnology, 37: 224-226 (2019)). CRISPR/Cas systems suitable for use in connection with the present disclosure are further described in, e.g., Marakova, K. S. and E. V. Koonin, Methods Mol. Biol., 1311: 47-75 (2015); Sander et al., Nat. Biotechnol., 32(4): 347-55 (2014); and Gootenberg et al., Science, 356(6336): 438-442 (2017)).

In other embodiments, the one or more nucleic acid molecules may be an RNA molecule. For example, the RNA molecule may be a messenger RNA (mRNA) sequence that encodes a protein. Alternatively, the RNA molecule may be non-protein coding. For example, the RNA molecule may comprise a nucleic acid sequence that is capable of inducing RNA interference (RNAi). The term “RNA interference” refers to a process in which RNA molecules inhibit gene expression or translation by neutralizing targeted mRNA molecules. To achieve an RNAi effect, for example, RNA having a double strand structure containing the same base sequence as that of the target mRNA may be used. Two types of small RNA molecules may induce RNAi: microRNA (miRNA) and small interfering RNA (siRNA). miRNA is a small non-coding RNA molecule (containing about 22 nucleotides) found in plants, animals and some viruses, which silences complementary target sequences by one or more of the following processes: (1) cleavage of the target mRNA strand into two pieces, (2) destabilization of the mRNA through shortening of its poly(A) tail, and (3) less efficient translation of the mRNA into proteins by ribosomes (Bartel D. P., Cell, 136 (2): 215-233 (2009); and Fabian et al., Annual Review of Biochemistry, 79: 351-79 (2010)). siRNA (also known as short interfering RNA or silencing RNA), is a class of double-stranded RNA molecules, typically 20-25 base pairs in length, which silence complementary target sequences by degrading mRNA after transcription, preventing translation (Dana et al., International Journal of Biomedical Science, 13(2):48-57 (2017); and Agrawal, et al., Microbiol. Mol. Biol. Rev., 67: 657-685 (2003)). siRNA can also act in RNAi-related pathways in an antiviral mechanism or play a role in the shaping of the chromatin structure of a genome. Any RNA molecule that is capable of silencing gene expression of a target gene may be used in connection with the present disclosure. In some embodiments, the RNA molecule is siRNA. In other embodiments, the RNA molecule may a long non-coding RNA (lncRNA). Long non-coding RNAs are a large and diverse class of transcribed RNA molecules with a length of more than 200 nucleotides that do not encode proteins. lncRNAs are thought to encompass nearly 30,000 different transcripts in humans, and account for the major part of the non-coding transcriptome. While the mechanism of action of lncRNAs is under investigation, lncRNAs appear to be important regulators of gene expression, and lncRNAs are thought to have a wide range of functions in cellular and developmental processes. lncRNAs may carry out both gene inhibition and gene activation through a range of diverse mechanisms (see, e.g., Kung et al., Genetics, 193(3): 651-666 (2013); and Marchese et al., Genome Biol., 18: 206 (2017)).

The nucleic acid molecule may comprise a nucleic acid sequence that is operatively linked to a promoter; however, nucleic acid sequences that lack a promoter are also within the scope of the present disclosure. As used herein, a “promoter” is a DNA sequence that directs the binding of RNA polymerase, thereby promoting RNA synthesis. A nucleic acid sequence is “operably linked” or “operatively linked” to a promoter when the promoter is capable of directing transcription of that nucleic acid sequence. A promoter can be native or non-native to the nucleic acid sequence to which it is operably or operatively linked. Techniques for operably linking sequences together are well known in the art. The promoter may be a ubiquitous promoter. The term “ubiquitous promoter,” as used herein, refers to a regulated or unregulated promoter that allows for continual transcription of its associated gene in a variety of cell types. Suitable ubiquitous promoters are known in the art and can be used in connection with the present disclosure. In other embodiments, the promoter may be a tissue-specific or cell-specific promoter. For example, the ubiquitous promoter may be a CAG promoter. The terms “tissue-specific promoter” and “cell-specific promoter,” as used herein, refer to a promoter that is preferentially activated in a given tissue or cell and results in expression of a gene product in the tissue or cell where activated. A tissue-specific or cell-specific promoter can be chosen based upon the target tissue or cell-type in which the nucleic acid sequence is to be expressed. In some embodiments, the nucleic acid molecule comprises a nucleic acid sequence that is operatively linked to an endothelial cell-specific promoter. Endothelial cell-specific promoters are known in the art (see, e.g., Schlaeger et al., Development, 121(4): 1089-98 (1995); Dai et al., J. Virol., 78(12): 6209-6221 (2004)), and include, for example the CDH5 promoter, which is the promoter of the human vascular endothelial-cadherin (CDH5) gene. (Gory et al., Blood, 93: 184-192 (1999); Huang et al., Circulation, 133: 1093-1103 (2016); and Prandini et al., Oncogene, 24: 2992-3001 (2005)), a Tie2 promoter (Fadel et al., Biochem J., 330 (Pt 1): 335-43 (1998)), or the 5′ endothelial enhancer of the stem cell leukemia locus (Gothert J R, et al., Blood, 104:1769-1077 (2004)).

The nucleic acid molecule, PLGA-PEG nanoparticle, and PEI may be combined in any desired ratio (in terms of weight or molarity) as determined by the practitioner. Exemplary ratios of PLGA-PEG nanoparticle:PEI include, but are not limited to, 2:1, 1:1, 1:2, 1:4, 1:6, 1:8, 1:10, and the like for large molecular weight PEI, or 1:20, 1:30, 1:40, 1:60, 1:120, 1:150, 1:180, 1:210, 1:250, 1:300 and the like for low molecular weight PEI. Exemplary ratios of nucleic acid molecule:PLGA-PEG/PEI nanoparticles include, but are not limited to. 1:1, 1:2, 1:3, 1:4, 1:5, 1:6, 1:10, and the like. In some embodiments, the ratio of nucleic acid molecule:PLGA-PEG nanoparticle:PEI may be about 1 μg:0.01-5 μg:0.1-100 μg, such as, for example, about 1 μg:0.375 μg:1.5 μg of PEI25k Da.

In some embodiments, the cargo molecule may be one or more small molecule compounds, with or without an associated nucleic acid molecule (as described herein). The term “small molecule compound,” as used herein, refers to refers to organic compounds, whether naturally-occurring or artificially created (e.g., via chemical synthesis) that have relatively low molecular weight and that are not proteins, polypeptides, or nucleic acids. Typically, small molecules have a molecular weight of less than about 1500 g/mol. Also, small molecules typically have multiple carbon-carbon bonds. A large number of naturally-occurring and synthetic small molecule compounds are known in the art and used as therapeutic agents against a wide variety of diseases. Examples of naturally-occurring small molecule compounds include, but are not limited to, penicillin, erythromycin, taxol, cyclosporin, and rapamycin. Examples of synthetic small molecule compounds include, but are not limited to, ampicillin, methicillin, sulfamethoxazole, and sulfonamides. Any suitable small molecule compound may be associated with the nanoparticle. In some embodiments, the PEI-coated PLGA-PEG nanoparticle can be used to co-deliver one or more nucleic acid molecules and one or more small molecule compounds. For example, a small molecule compound may be encapsulated within a PLGA-PEG nanoparticle, which may then be coated with PEI and associated with a nucleic acid molecule of interest.

The disclosure also provides a method of delivering one or more cargo molecules to a cell, which comprises contacting the cell with the nanoparticle composition described herein. Descriptions of PLGA-PEG nanoparticles, PEI, cargo molecules, nucleic acid molecules, small molecule compounds, and components thereof described herein also are applicable to those same aspects of the aforementioned method of delivering one or more nucleic acid molecules to a cell.

The cell may be contacted with the nanoparticle composition in vitro or in vivo. The term “in vivo” refers to a method that is conducted within healthy or diseased living organisms in their intact state, while an “in vitro” method is conducted using components of an organism that have been isolated from its usual biological context. When the cell is contacted with the nanoparticle in vitro, the cell desirably is a eukaryotic cell. Suitable eukaryotic cells are known in the art and include, for example, insect cells, and mammalian cells including immortal cell lines and cancer cells. Suitable insect cells are described in, for example, Kitts et al, Biotechniques, 14: 810-817 (1993); Lucklow, Curr. Opin. Biotechnol, 4: 564-572 (1993); and Lucklow et al, J. Virol, 67: 4566-4579 (1993), and include Sf-9 and HI5 (Invitrogen, Carlsbad, Calif.).

In some embodiments, the cell is a mammalian cell. A number of suitable mammalian host cells are known in the art, and many are available from the American Type Culture Collection (ATCC, Manassas, Va.). Examples of suitable mammalian cells include, but are not limited to, fibroblasts, Chinese hamster ovary cells (CHO) (ATCC No. CCL61), CHO DHFR-cells (Urlaub et al, Proc. Natl. Acad. Sci. USA, 97: 4216-4220 (1980)), mouse Hepa1c1c7 (ATCC No. CRL-2026), Neuro-2a (ATCC No. CCL-131), human embryonic kidney (HEK) 293 or 293T cells (ATCC No. CRL1573), and 3T3 cells (ATCC No. CCL92), and various cancer lines. Other suitable mammalian cell lines are the monkey COS-1 (ATCC No. CRL1650) and COS-7 cell lines (ATCC No. CRL1651), as well as the CV-1 cell line (ATCC No. CCL70). The cell may be a fibroblast, a neuron cell, a hepatocyte, or a cardiomyocyte. In certain embodiments, the cell is an endothelial cell, such as an endothelial cell of the pulmonary vasculature, cardiac vasculature, aortic vasculature, or the skeletal muscular vasculature. Methods for selecting suitable mammalian host cells and methods for transformation, culture, amplification, screening, and purification of cells are known in the art. In certain embodiments, the cell is a human cell, such as a human endothelial cell or cancer cell.

When the cell is contacted with the nanoparticle composition in vivo, the composition desirably is a pharmaceutically acceptable (e.g., physiologically acceptable) composition, which comprises the nanoparticle and the associated one or more nucleic acid molecules, with or without a carrier, preferably a pharmaceutically acceptable (e.g., physiologically acceptable) carrier. Any suitable carrier can be used within the context of the disclosure, and such carriers are well known in the art. The choice of carrier will be determined, in part, by the particular site to which the composition may be administered and the particular method used to administer the composition. The composition optionally can be sterile. The composition can be frozen or lyophilized for storage and reconstituted in a suitable sterile carrier prior to use. The compositions can be generated in accordance with conventional techniques described in, e.g., Remington: The Science and Practice of Pharmacy, 21st Edition, Lippincott Williams & Wilkins, Philadelphia, Pa. (2001).

The composition may be administered to an animal, such as a mammal, particularly a human, using standard administration techniques, including intravenous, subcutaneous, pulmonary, transdermal, intramuscular, intranasal, buccal, sublingual, suppository, or inhalational administration. The composition preferably is suitable for parenteral administration. The term “parenteral,” as used herein, includes intravenous, intramuscular, intracardial, subcutaneous, inhalational, rectal, and vaginal administration. In other embodiments, the composition may be administered to a mammal using peripheral systemic delivery by intravenous or subcutaneous injection.

The following examples further illustrate the invention but, of course, should not be construed as in any way limiting its scope.

### EXAMPLES

The cardiovascular endothelium is a monolayer of endothelial cells lining the luminal surface of all blood vessels. Among its vital functions are regulation of vascular permeability and retention of blood cells in the circulation (Cines et al., Blood 91, 3527-3561 (1998)). Because of its permeability properties, the endothelial-cell layer permits free exchange of small crystalloid and nutrient molecules across vessel walls, but is highly restrictive to protein, thus enabling formation of an oncotic pressure gradient, which counter-balances the hydrostatic pressure generated by the pumping action of the heart to achieve tissue-fluid balance at physiological vascular pressures (Mehta, D. and Malik, A. B., Physiol Rev, 86: 279-367 (2006)). The endothelial layer also plays a key role in vasomotor regulation in all vascular beds and is critically involved in the regulation of immune and coagulation responses with precise localization to the area of need (Munoz-Chapuli, Evol. Dev., 7: 351-358 (2005)). The endothelium is responsible for the antithrombotic surface of normal blood vessels, and provides a nearly frictionless conduit for blood flow, thus minimizing the energy required to propel the blood (Monahan-Earley et al., Thromb. Haemost., 11 Suppl 1: 46-66 (2013)). Under adverse conditions (as for example, infection, tissue necrosis, immune reactions, or hypercholesterolemia) endothelial cells are activated, leading to inflammation and endothelial barrier disruption (increased vascular permeability, edema fluid formation, release of proinflammatory cytokines, and leukocyte extravasation) (Ware., L. B. and Matthay, M. A., N. Engl. J. Med., 342: 1334-1349 (2000)).

Endothelial dysfunction figures prominently in the etiology of atherosclerosis, the pathological process underlying the major cardiovascular diseases (myocardial infarction, stroke, coronary artery disease, and peripheral artery disease). The ability to genetically modify the endothelia of the cardiovascular system has been challenging thus far considering the lack of a delivery system capable of targeting endothelia other than the liver for genome editing.

The experiments described below demonstrate that nanoparticle-mediated delivery of an all-in-one-CRISPR plasmid DNA expressing Cas9 under the control of the human CDH5 promoter (endothelial cell-specific) results in highly efficient genome editing specifically in endothelial cells (ECs) of the cardiovascular system including heart, lung, and aorta in adult mice, which leads to disruption of gene expression and a phenotype mimicking that of genetic knockout mice. The experiments described below also demonstrate that nanoparticle delivery of plasmid DNA results in increased gene expression in vascular endothelial cells.

### Example 1

This example demonstrates a method of highly efficient gene transfection in various cells which results in highly efficient in vitro genome editing using the nanoparticle composition described herein.

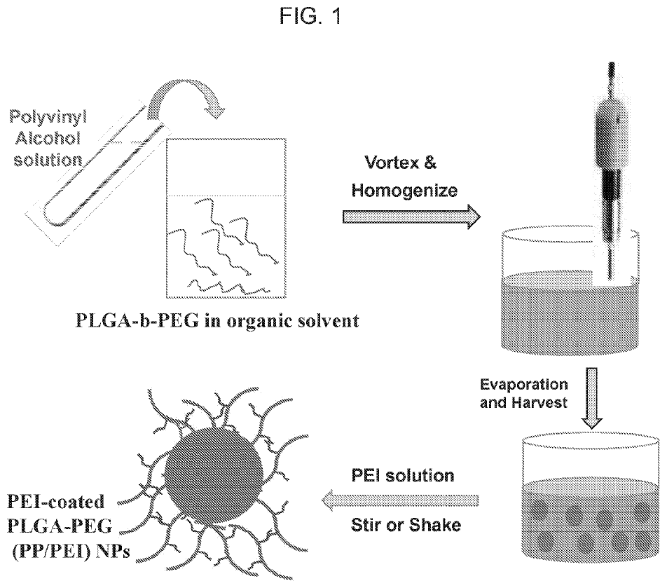

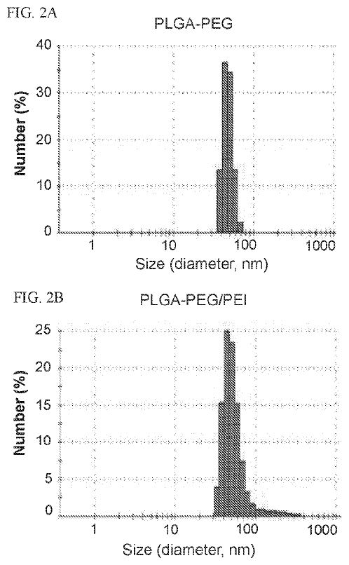

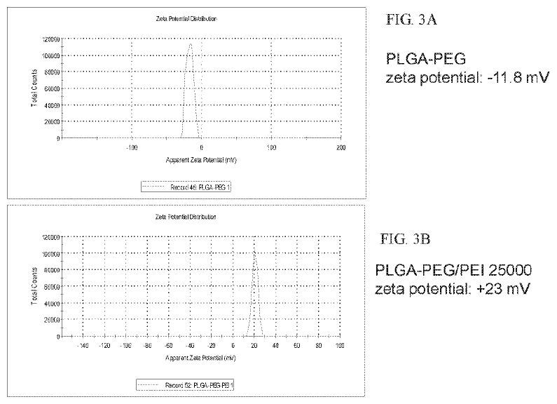

The frequency of genome editing by CRISPR/Cas9 is dependent on guide RNA (gRNA) potency and expression of Cas9 (Mali, P. et al., Science, 339: 823-826 (2013)). To establish a high-throughput method for identification of potent gRNAs, a PLGA-PEG/PEI (PPP) nanoparticle was developed for high efficient transfection by a two-step method. First, PLGA-PEG nanoparticles were formulated by emulsification and evaporation as previously described (Kleinstiver et al., Nature, 529: 490-495 (2016)) with modifications. Briefly, 80 mg of PEG-PLGA (Sigma, USA) were dissolved in 8 ml of dichloromethane and homogenized for 40 seconds to form the oil phase emulsification. The oil phase emulsification was combined with 20 ml polyvinyl alcohol (1% w/w, Sigma, USA) and homogenized for another 40 seconds to form the second water phase emulsification, which was added to 100 ml water and stirred for 6 hours to make organic reagent evaporate and nanoparticles harden. The PLGA-PEG nanoparticles were harvested by centrifugation at 12,000 rpm for 20 minutes and washed 3 times with ultrapure water. Second, the synthesized PLGA-PEG nanoparticles were mixed with polyethyleneimine (PEI) at 1:4 (PLGA-PEG:PEI) ratio and incubated at room temperature for 72 hours. Following centrifugation at 19,000 g for 20 minutes, the supernatant was collected, the size of the harvested PLGA-PEG-PEI (PP/PEI) nanoparticles was estimated by dynamic laser scattering using a Zetasizer Nano ZS (Malvern Instruments, UK). Preparation of the PLGA-PEG/PEI nanoparticles is illustrated schematically in FIG. 1. The size of PLGA-PEG nanoparticles was 35-70 nm (90%) (FIG. 2A), while the size of PP/PEI nanoparticles was 30-200 nm (95% was 30-100 nm) (FIG. 2B). The zeta potential of PLGA-PEG and PP/PEI nanoparticles was −12 mV (FIG. 3A) and +23 mV (FIG. 3B), respectively.

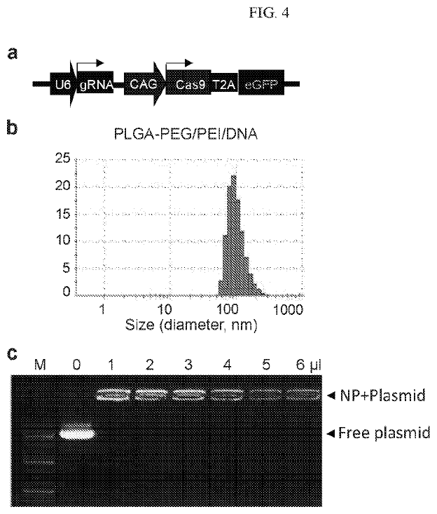

Next, the transfection efficiency of an all-in-one CRISPR^{CAG} plasmid DNA (Ran et al., Nat Protoc., 8: 2281-2308 (2013)) expressing Cas9 under the control of the chicken Actb promoter with a CMV enhancer (CAG) and gRNA driven by the U6 promoter was tested. The plasmid is illustrated schematically in FIG. 4A. Preparation of the CRISPR/Cas9/gRNA plasmid DNA was performed as described previously (Wang et al., Chem. Rev., 117, 9874-9906 (2017)). Briefly, the single complementary DNA oligonucleotides corresponding to the gRNA sequence were commercially synthesized (Integrated DNA Technologies). After phosphorylation and annealing, the paired double-stranded DNA oligo was cloned into the BbsI linearized plasmid pSpCas9(BB)-2A-GFP14. Positive clones containing the gRNA-encoded DNA sequence were identified by DNA sequencing. For endothelial cell-specific genomic editing, the CAG promoter (chicken Actb promoter with CMV enhancer) in pSpCas9(BB)-2A-GFP plasmid was replaced with the human CDH5 promoter (Prandini et al., Oncogene, 24: 2992-3001 (2005); and Zhao et al., J Clin Invest., 116: 2333-2343 (2006)) using Kpn I and Age I restriction enzyme sites (CRISPR^{CDH5}). All gRNA sequences are listed in Table 1. The characterized nanoparticles were mixed with CRISPR plasmid DNA at the optimized ratio of 1 μg plasmid DNA to 3 μl PP/PEI nanoparticles and kept at room temperature for 10 minutes before use.

TABLE 1

| gRNA Name | Sequence (5′-3′) |
|---|---|
| Pik3cg-gRNA-1 | ACCGTACCACGACAGTGCGC |
| | (SEQ ID NO: 1) |
| Pik3cg-gRNA-2 | ATCTGGCCAGCGCACTGTCG |
| | (SEQ ID NO: 2 |
| Pik3cg-gRNA-3 | AGCCTCGCAGGTACGCCTCC |
| | (SEQ ID NO: 3) |
| Pik3cg-gRNA-4 | ACTAAAAGCCGGTACCCTGG |
| | (SEQ ID NO: 4) |

The PP/PEI nanoparticles formed a complex with the CRISPR plasmid DNA with size of 50-300 nm (95% are 50-2000 nm), as shown in FIG. 4B, and the PP/PEI nanoparticles efficiently neutralized the plasmid's negative charge, as shown in FIG. 4C.

Hepa-1c1c7 (ATCC®) were maintained in DMEM with 10% FBS, 100U/ml penicillin, and 100 μg/ml streptomycin. The all-in-one CRISPR^{CMG} plasmid DNA was transfected to Hepa-1c1c7 cells (cell density 50-70%) in complete medium, i.e., without starvation using nanoparticles with the optimized ratio of 1 μg plasmid to 4 μl nanoparticles. At 72 h post-transfection, the transfected cells were collected for total RNA and genomic DNA (gDNA) isolation. cDNA was synthesized from total RNA using reverse transcriptase. The cDNA and gDNA were used for quantitative real-time PCR to identify the highly potent gRNAs. The gDNA containing the gRNA-target sequence was also amplified by PCR and the PCR product was then used for sequencing to determine insertions/deletions (“indels”) using Tide software analysis.

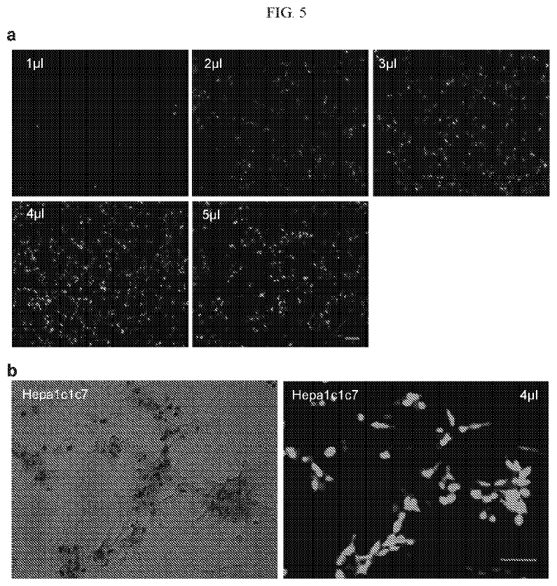

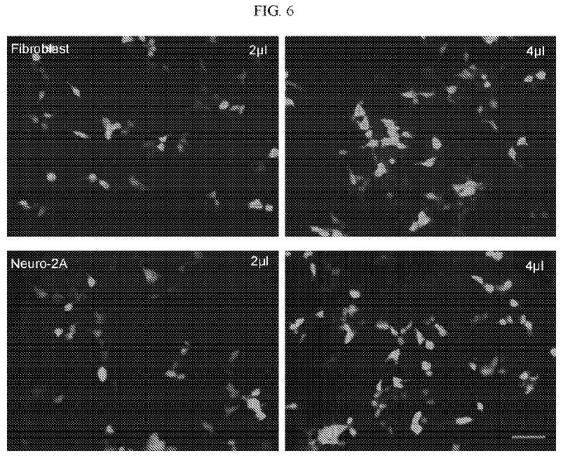

The PP/PEI nanoparticle-CRISPR plasmid complex resulted in greater than 90% transfection efficiency in Hepa-1c1c7 cells, as shown in FIGS. 5A and 5B. The transfection efficiency in primary cultures of mouse fibroblasts and Neuro-2A cell lines also was tested, and PP/PEI nanoparticles induced great than 90% transfection efficiency in both cell types, as shown in FIGS. 6A-6D.

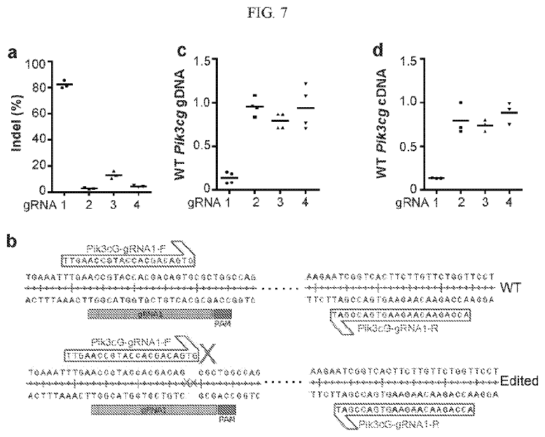

The high transfection efficiency led to directly determining the efficiency of genome editing by measuring nucleic acid indels without further purification or enrichment of transfected cells. Sanger sequencing decomposition analysis revealed that gRNA1 targeting the mouse Pik3cg gene (see Table 1), which encodes the p110γ isoform of PI3K, caused greater than 80% genome editing in cultured cells whereas other gRNAs induced less than 20% genome editing, as shown in FIG. 7A. The indels are generated through the deletion or insertion of bases during non-homologous end joining (NHEJ) repair after Cas9 cleavage-induced DNA double-strand breaks (Shalem et al., Nat Rev Genet 16, 299-311 (2015)). Given that the frequency of indels formed by base deletion is 3-4 fold greater than by base insertion, and that the deleted bases are often located upstream of the Cas 9 cleavage site (Wang et al., supra), a PCR primer was designed comprising the deleted bases for quantitative real time PCR screening, as the forward primer will not amplify the mutated DNA with deletions due to 3′ mismatch but will amplify the wild type DNA (see FIG. 7B).

Quantitative PCR analysis demonstrated that gRNA1 induced an 80% reduction in wild type genomic DNA. The other gRNAs induced less than a 20% reduction. Similarly, gRNA1 induced an 80% reduction in the wild type cDNA converted from Pik3cg mRNA, as shown in FIGS. 7C and 7D. These data are consistent with the data from classical Sanger sequencing decomposition analysis. Using a similar quantitative PCR screening method, potent gRNAs were identified for 10 other genes, including Vegfr2 (data not shown).

Thus, the quantitative PCR-based screening method coupled with PP/PEI nanoparticle-mediated high efficiency transfection is a relatively simple and efficient method for identification of potent gRNAs in vitro.

### Example 2

This example demonstrates a method of highly efficient cell-specific in vivo genome editing in mouse lungs using the nanoparticle composition described herein.

C57BL/6J mice at 3-5 months of age (The Jackson Laboratory) were used for CRISPR/Cas9-mediated in vivo genome editing. All mice were bred and maintained in the Association for Assessment and Accreditation of Laboratory Animal Care-accredited animal facilities at the Stanley Manne Children's Research Institute according to National Institutes of Health guidelines. All animal experiments were performed in accordance with protocols approved by Northwestern University Institutional Animal Care and Use Committee.

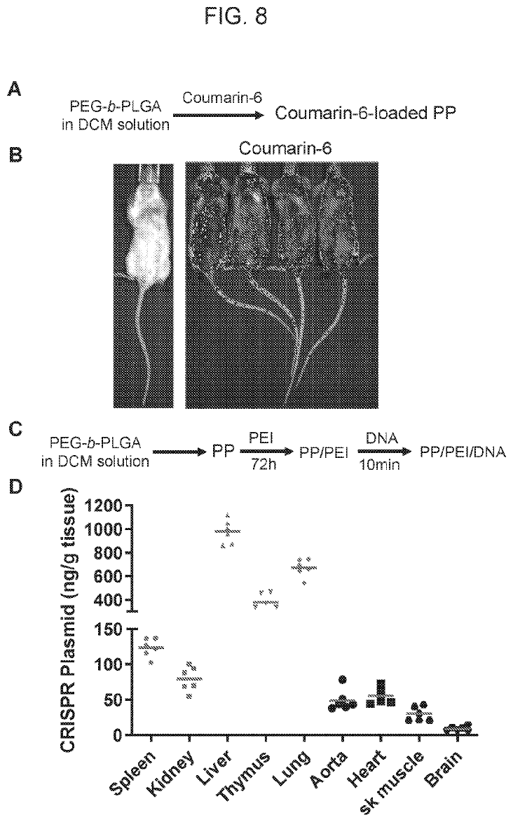

To determine biodistribution of the PLGA-PEG (PP) nanoparticle, fluorescent dye coumarin-6-loaded PP nanoparticles were administrated to adult mice through retro-orbital injection. 5 hours post-administration, fluorescent tomography by IVIS imaging of live mice demonstrated that the PP nanoparticles were distributed throughout the whole body without specific accumulation in the liver and other organs (FIGS. 8A and 8B). Then, CRISPR plasmid DNA-loaded PP/PEI nanoparticles were administered to adult mice. At 8 h post-administration, various organs were collected for quantification of plasmid DNA accumulation. PCR analysis shows that plasmid DNA is highly enriched in liver, lung, thymus as well as in spleen, kidney, heart, aorta and skeletal muscle (FIGS. 8C and 8D).

To investigate the possibility that PP/PEI nanoparticles may deliver the CRISPR plasmid DNA to the whole cardiovascular system and thereby induce in vivo genome editing, the CAG promoter in the CRISPR^{CAG} plasmid DNA was replaced with the 3.5 kb human CDH5 promoter which is endothelial cell (EC)-specific (Gory et al., supra, Huang et al., supra, and Prandini et al., supra). CRISPR^{CDH5} plasmid DNA was mixed with PP/PEI nanoparticles at a ratio of 1 μg of plasmid DNA:3 μl nanoparticles or nanoparticle made with PLGA only (PLGA) or PEI only (1× or 3× amount of PEI used in the PP/PEI nanoparticles), and kept at room temperature for 10 min before use. Each mouse was given 40 μg plasmid DNA via intravenous (retro-orbital) injection. 7-10 days after nanoparticle delivery, mice were used for experiments.

The mice were euthanized for tissue collection (lung, heart, abdominal aorta, and hindlimb skeletal muscle) 7 days after administration of PP/PEI-CRISPR^{CDH5} plasmid DNA, and ECs and non-ECs were then isolated. Briefly, after perfused free of blood with PBS, lung tissue, heart, abdominal aorta, or skeletal muscle was cut into small pieces, and then incubated with 1 mg/ml collagenase A (Roche Applied Science) for 1 h at 37° C. in a shaking water bath (200 rpm). After digestion, the tissue was dispersed to a single cell preparation using the gentleMACS™ Dissociator (Miltenyi Biotec) with lung program 2 (which also works well with heart and aorta). The cells were then filtered using a 40 μm Nylon cell strainer and blocked with 20% FBS for 30 min. After 15 min incubation with Fc blocker (1 μg/10⁶ cells, BD Biosciences), the cells were incubated with anti-CD31 (1:1000, BD Biosciences) for 30 min at room temperature. After washing twice, the immunostained cells in 1 ml PBS were added with 50 μl pre-washed Dynabeads conjugated with anti-rat IgG secondary antibody, and incubated for 30 min at room temperature. The cells were then subjected to magnetic purification. After washing twice, the cells were used for experiments. Flow cytometry analysis demonstrated that the purity of ECs isolated by magnetic sorting was about 80%. Non-ECs were collected from the wash-through cells after 2 times anti-CD31 incubation.

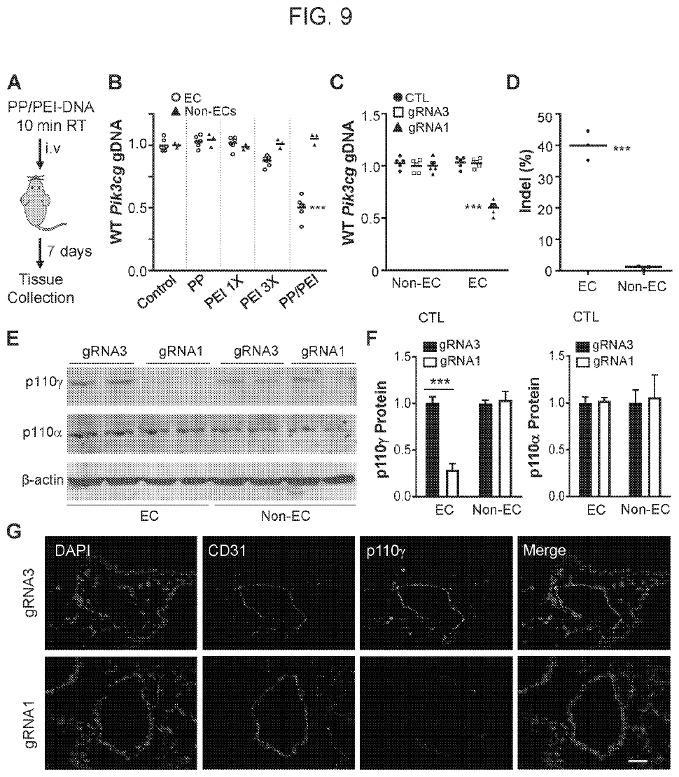

Quantitative PCR analysis was then performed. Briefly, mouse tissues were lysed in Trizol reagent (ThermoFisher Scientific) using the TissueLyser (Qiagen) and total RNA was isolated and purified using the RNeasy mini kit including DNase I digestion (Qiagen) according to manufacturer's instructions. Total RNA from cultured cells was isolated directly using the RNeasy mini kit. Following conversion of RNA to cDNA with reverse transcriptase (Applied Biosystems), SYBR Green-based quantitative real time PCR analyses (Roche Applied Science) were performed with the 7500 fast Real-Time PCR System (Thermo Fisher Scientific). All qPCR primers are listed in Table 2. The results of qPCR demonstrated that wild type Pik3cg genomic DNA was decreased by about 50% in ECs isolated from lungs of PP/PEI/gRNA1-transduced mice compared to control mice, as shown in FIG. 9B. However, the PLGA/gRNA1 plasmid nanoparticle or PEI/gRNA1 plasmid mixture has no or only minimal effect on genome editing. Thus, only PP/PEI nanoparticle delivery of CRISPR plasmid can induce high efficiency of genome editing in mice. Next, we addressed the efficiency of PP/PEI delivery of CRISPR plasmid DNA in genome editing in adult mice. The results of qPCR demonstrated that wild type Pik3cg genomic DNA was decreased by about 45% in ECs isolated from lungs of gRNA1-transduced mice compared to control- or gRNA3-transduced mice, as shown in FIG. 9C. There were no changes in non-ECs. Considering that deletions were 3-4-fold greater than insertions and cell purity was 80%, it was estimated that this strategy induced at least 70% indels in lung ECs. Sanger sequencing decomposition analysis demonstrated that the genome editing rates were as high as 40% in isolated lung ECs, whereas there was no genome editing in non-ECs, as shown in FIG. 9D.

TABLE 2
PCR primer sequences for genome editing analysis
Name Forward (5-3) Reverse (5-3)
qPCR primers for Pik3cg cDNA

| | | |
|---|---|---|
| gRNA1 | TTGAACCGTACCACGACAGTG | CTCCAGGATCACAGCGAACCT |
| | (SEQ ID NO: 5) | (SEQ ID NO: 6) |
| gRNA2 | TTAACCATGCAGCTCCTGGAC | GAATCTGGCCAGCGCACTG |
| | (SEQ ID NO: 7) | (SEQ ID NO: 8) |
| gRNA3 | TTGAACCGTACCACGACAGTG | AGCCTCGCAGGTACGCC |
| | (SEQ ID NO: 9) | (SEQ ID NO: 10) |
| gRNA4 | TGGACTAAAAGCCGGTACCC | TCGTTGGATAGGACTGTGGG |
| | (SEQ ID NO: 11) | (SEQ ID NO: 12) |

qPCR primers for Pik3cg genomic DNA

| | | |
|---|---|---|
| gRNA1 | TTGAACCGTACCACGACAGTG | ACCAGAACAAGAAGTGACCGAT |
| | (SEQ ID NO: 13) | (SEQ ID NO: 14) |
| gRNA2 | GAACTGTGGGTTTCCCCCAT | GAATCTGGCCAGCGCACTG |
| | (SEQ ID NO: 15) | (SEQ ID NO: 16) |
| gRNA3 | TTGAACCGTACCACGACAGTG | AGCCTCGCAGGTACGCC |
| | (SEQ ID NO: 17) | (SEQ ID NO: 18) |
| gRNA4 | TGGACTAAAAGCCGGTACCC | TGGTGCTAGTGATGAGAGGGT |
| | (SEQ ID NO: 19) | (SEQ ID NO: 20) |

PCR primers for Pik3cg Indel analysis

| | | |
|---|---|---|
| gRNA-1-2-3 | TGCTAAGCAAGTGCCTCCTC | CTTCTCTGCCGAGAGCGATT |
| | (SEQ ID NO: 21) | (SEQ ID NO: 22) |
| gRNA4 | TGGAATGGTTAGATCCCCAA | TCAGGGAACTTCACCTATGGA |
| | (SEQ ID NO: 23) | (SEQ ID NO: 24) |

qPCR primer for Vegfr2 cDNA

| | | |
|---|---|---|
| gRNA1 | GGCGGTGGTGACAGTATCTT | CGTCCCGGTACGAGCACT |
| | (SEQ ID NO: 25) | (SEQ ID NO: 26) |
| gRNA2 | GGCGGTGGTGACAGTATCTT | CGGTGATGTACACGATGCCA |
| | (SEQ ID NO: 27) | (SEQ ID NO: 28) |
| gRNA3 | GGCAAATACAACCCTTCAGATTACT | CGTCCCGGTACGAGCACT |
| | (SEQ ID NO: 29) | (SEQ ID NO: 30) |
| gRNA4 | ACTGGAGCCTACAAGTGCTCG | CGGTGATGTACACGATGCCA |
| | (SEQ ID NO: 31) | (SEQ ID NO: 32) |
| gRNA5 | AAACTGTGGTGATCCCCTGC | GAGAGTAAAGCCTATCTCGCTGT |
| | (SEQ ID NO: 33) | (SEQ ID NO: 34) |
| gRNA6 | AGAATTTCCTGGGACAGCGA | TTGCCTCACAGAAGACCATGC |
| | (SEQ ID NO: 35) | (SEQ ID NO: 36) |

qPCR primer for Vegfr2 genomic DNA

| | | |
|---|---|---|
| gRNA1 | GGCGGTGGTGACAGTATCTT | CGTCCCGGTACGAGCACT |
| | (SEQ ID NO: 37) | (SEQ ID NO: 38) |
| gRNA2 | GATGTCCGCATTCATGCAAGT | CGGTGATGTACACGATGCCA |
| | (SEQ ID NO: 39) | (SEQ ID NO: 40) |
| gRNA3 | AGTGGAAATTGTTGTGACCTCAG | AGTGGAAATTGTTGTGACCTCAG |
| | (SEQ ID NO: 41) | (SEQ ID NO: 42) |
| gRNA4 | ACTGGAGCCTACAAGTGCTCG | ACGACATTGGAAGCAGACGG |
| | (SEQ ID NO: 43) | (SEQ ID NO: 44) |
| gRNA5 | CGTTGAATTTAGACAGTGCTGGG | GAGAGTAAAGCCTATCTCGCTGT |
| | (SEQ ID NO: 45) | (SEQ ID NO: 46) |
| gRNA6 | AGAATTTCCTGGGACAGCGA | TTGCCTCACAGAAGACCATGC |
| | (SEQ ID NO: 47) | (SEQ ID NO: 48) |

PCR primer for Vegfr2 Indel analysis

| | | |
|---|---|---|
| gRNA, 1-4 | GAGCGTGATATCCTTGGTCCC | GTCTCCATAGCTCCTGTCGG |
| | (SEQ ID NO: 49) | (SEQ ID NO: 50) |
| gRNA3 | GGGCAAAGTCAATCCCACCT | AGCCCAGAACATCAAGCCAG |
| | (SEQ ID NO: 51) | (SEQ ID NO: 52) |
| gRNA-2-5-6 | ATGTGCAGACTAGCCTGTGG | AGAGGAGAAACCCCAGTTGC |
| | (SEQ ID NO: 53) | (SEQ ID NO: 54) |

To determine changes in protein levels, Western blotting and immunofluorescent staining were performed. To collect tissue lysates for Western blotting, mouse tissues were lysed in RIPA buffer using the TissueLyser (Qiagen). 30 μg of lysates/lane were loaded for Western blot analysis with the following antibodies: anti-p110γ (1:1000, Cellular Signaling Technology), anti-p110α (1:1000, Abcam), and anti-β-actin (1:3000, BD Biosciences). p110γPI3K protein expression in lung ECs of gRNA1-trasnduced mice was markedly diminished compared to that in lung ECs of gRNA3-transduced mice, as shown in FIGS. 9E and 9F. However, expression of the p110α isoform of PI3K was not affected and p110γPI3K expression in non-ECs was similar.

For immunofluorescent staining, cryosections (3-5 μm) of mouse tissues (perfused free of blood with PBS) were fixed with 4% paraformaldehyde and then immunostained with anti-p110γ antibody (1:200, Cellular Signaling Technology). Sections also were immunostained with anti-CD31 (1:100, BD Science) to identify vascular ECs. Nuclei were counterstained with DAPI (Prolong Gold Antifade Mountant with DAPI, ThermoFisher Scientific). Sections were imaged with a confocal microscope system (LSM 880). Immunofluorescent staining revealed diminished p110γ expression in pulmonary vascular ECs in gRNA1-transduced mice but not in gRNA3-transduced mice, as shown in FIG. 9G. These data demonstrate that the nanoparticle composition described herein is capable of mediating cell- and gene-specific genome editing in adult mice.

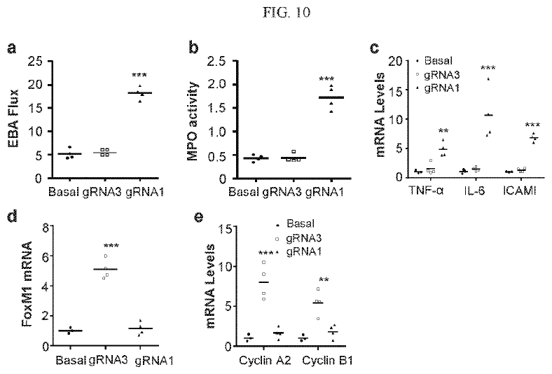

Previous work has shown that genetic deletion of Pik3cg in lung ECs interfered with vascular repair and resolution of inflammation following LPS-induced inflammatory vascular injury (Huang et al., Circulation, 133: 1093-1103 (2016)). Thus, the possibility that CRISPR-mediated genome editing of Pik3cg would result in a similar phenotype in adult mice was investigated. At 7 days following nanoparticle/CRISPR^{CDH5} plasmid DNA delivery, mice were challenged with the endotoxin lipopolysaccharide (LPS), the major component of the outer membrane of Gram-negative bacteria to induce inflammatory vascular injury. Specifically, LPS (E. coli 055:B5, Santa Cruz) was administered i.p. to mice at a dose of 2.5 mg/kg body weight in PBS (10 μl/g). At 72 hours post-LPS challenge (when WT mice were fully recovered), lungs were collected for determination of vascular permeability and inflammation. An assay measuring pulmonary transvascular flux of Evans blue-conjugated albumin (EBA) was carried out as described previously (Zhao et al., J. Clin. Invest., 116: 2333-2343 (2006)). Briefly, Evans blue dye (Sigma) was dissolved in PBS at 15 mg/ml with slow shaking at room temperature for 3 hours and the solution was collected after centrifugation. Bovine serum albumin (fraction V, Sigma) was also dissolved in PBS (8 mg/ml) and purified with charcoal (Sigma) by mixing 150 mg albumin with 300 mg charcoal in 12.5 ml PBS. Following vortexing (30 sec, 10 times), the solution was incubated for 1 h at room temperature with slow shaking and then centrifuged at full speed (13,000 rpm) for 5 min. The supernatant was collected and centrifuged for another 5-6 times until there were no particles in the supernatant. Evans blue and albumin solutions were mixed at a 1:2 ratio and incubated for 45 min with slow shaking at room temperature and then sterile-filtered through a 0.22 μm syringe filter. EBA (20 mg/kg BW) was retro-orbitally injected into mice 40 min before tissue collection. Lungs were perfused free of blood with PBS, blotted dry, weighed and snap frozen in liquid nitrogen. The right lung was homogenized in 0.5 ml PBS and incubated with 1 ml formamide at 60° C. for 18 h. The homogenate was then centrifuged at 21,000×g for 10 min and the optical density of the supernatant was determined at 620 nm and 740 nm. Extravasated EBA in lung homogenates was expressed as micrograms of Evans blue dye per g lung tissue. The EBA flux assay showed a persistent increase in lung vascular permeability at 72 h post-LPS in gRNA1-transduced mice, whereas permeability recovered to the basal value in gRNA3-transduced mice (see FIG. 10A), thus indicating impaired vascular repair in gRNA1-transduced mice.

Lung inflammation was assessed by measuring myeloperoxidase (MPO) activity, which is indicative of neutrophil sequestration. Briefly, lung tissues perfused free of blood with PBS were homogenized in 5 mM (0.5 ml) phosphate buffer (pH 6.0) and then centrifuged at 21,000×g for 10 minutes at 4° C. The pellets were resuspended in phosphate buffer containing 0.5% hexadecyl trimethylammonium bromide (Sigma) and subjected to a cycle of freezing and thawing. The pellets were then homogenized and the homogenates were centrifuged again. The supernatants were assayed for MPO activity 21,33 by mixing 50 μl of sample, 75 μl of 0.015% H₂O₂, and 15 μl of O-dianisidine dihydrochoride solution (16.7 mg/ml) in 1.38 ml of phosphate buffer, and reading absorbance at 460 nm every 20 sec for 3 minutes. Results are expressed as ΔOD460/min/g lung tissue. Lung inflammation was not resolved in gRNA1-transduced mice at 72 h post-LPS in contrast to gRNA-3-transduced mice, as shown in FIGS. 10B and 10C, which is consistent with defective vascular repair in gRNA1-transduced mice.

Expression of the reparative transcription factor FoxM1, which is downstream of p110γ signaling and mediates EC proliferation and re-annealing of adherens junctions for vascular repair (Zhao et al., J Clin Invest, 116: 2333-2343 (2006), Mirza et al., Exp Med, 207: 1675-1685 (2010); and Huang et al., Circulation, 133:1093-1103 (2016)) during the recovery phase, was induced in gRNA3-transduced mice but not in gRNA1-transduced mice (see FIG. 10D), further demonstrating the inhibition of p110γ signaling in gRNA1-transduced mouse lungs. Accordingly, expression of the FoxM1 target genes Ccna2 and Ccnb1, which are essential for cell cycle progression, was not induced in gRNA1-transduced mouse lungs, as shown in FIG. 10E.

The results of this example demonstrate that nanoparticle delivery of CRISPR^{CDH5} plasmid DNA induces highly efficient genome editing in lung ECs leading to diminished p110γ expression, which in turn results in defective vascular repair and resolution of inflammation as seen in Pik3cg^{-/-} mice (Huang et al., Circulation, 133:1093-1103 (2016)).

### Example 3

This example demonstrates highly efficient genome editing in the systemic and peripheral vascular endothelial cells following nanoparticle delivery of CRISPR^{CDH5} plasmid DNA.

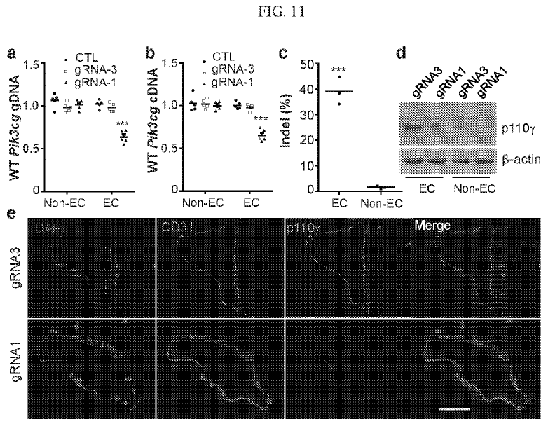

Quantitative PCR revealed about a 40% decrease in wild-type Pik3cg genomic DNA and cDNA selectively in ECs obtained from hearts of gRNA1-transduced but not gRNA3-transduced mice, as shown in FIGS. 11A and 11B. Sanger sequencing decomposition analysis indicated that the genome editing rate was 40% in ECs isolated from gRNA1-transduced mouse hearts, as shown in FIG. 11C. No genome editing was observed in non-ECs. Western blotting demonstrated diminished p110γ expression in ECs isolated from gRNA1-transduced mouse hearts compared to those of gRNA3-transduced mice, as shown in FIG. 11D. There was no difference in p110γ expression in non-ECs obtained from gRNA1 or gRNA3-transduced mouse hearts. Immunofluorescent staining also showed a marked decrease in p110γ expression in ECs in gRNA1-transduced mouse hearts, as shown in FIG. 11E.

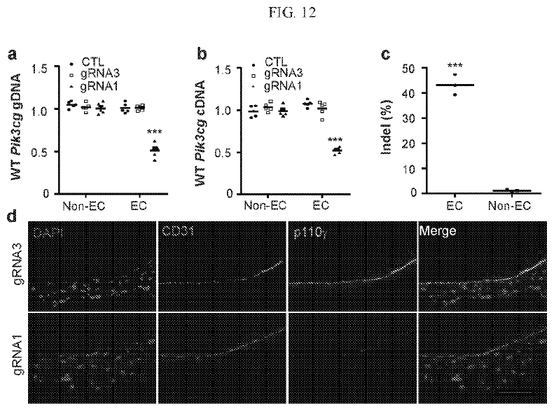

In the abdominal aorta, a 50% reduction in wild-type genomic DNA or cDNA was detected (see FIGS. 12A and 12B) and greater than 40% indels (FIG. 12C) was observed in ECs isolated from the aorta of gRNA1-transduced mice. The genome editing was selectively induced in ECs, as shown in FIGS. 12A-12C. Immunofluorescent staining revealed diminished p110γ expression in ECs obtained from the aorta of gRNA1-transduced mice, but not in gRNA3-transduced mice (FIG. 12D). Together, these data demonstrate highly efficient genome editing in ECs of the aortic vascular ECs by nanoparticle delivery of CRISPR^{CDH5} plasmid DNA in adult mice.

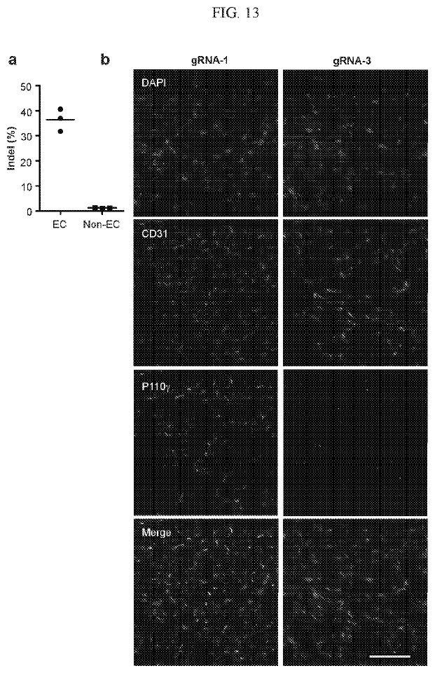

The efficiency of genome editing in the peripheral vasculature also was assessed. As shown in FIG. 13A, Sanger sequencing decomposition analysis revealed a 35% genome editing rate in ECs isolated from gRNA1-transduced mouse thigh muscular vessels but not in non-ECs. Immunofluorescent staining demonstrated a marked decrease in protein expression of p110γPI3K in peripheral vascular ECs of gRNA1-transduced mice, as shown in FIG. 13B.

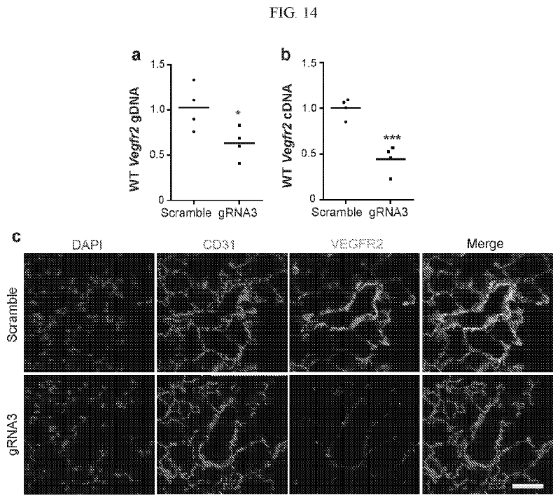

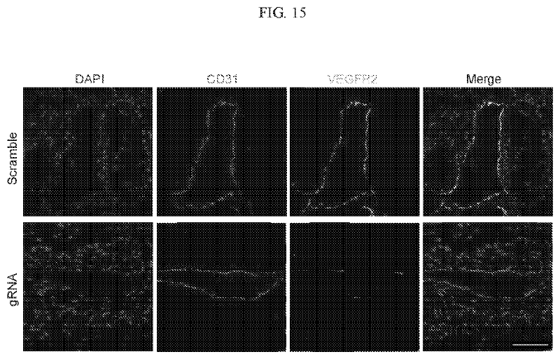

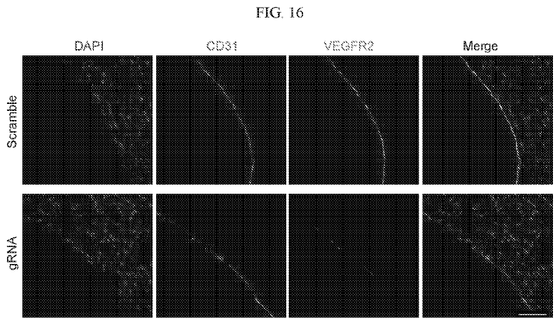

The in vivo genome editing efficiency of Vegfr2 gRNA also was assessed. As described above, lung tissues were collected 7 days after nanoparticle delivery of CRISPR^{CDH5}, and quantitative PCR was performed. Quantitative PCR analysis demonstrated a 38% reduction in wild type Vegfr2 genomic DNA and a 50% reduction in wild type Vegfr2 cDNA converted from Vegfr2 mRNA in whole lung tissue of gRNA3-transduced mice (FIGS. 14A and 14B). Immunofluorescent staining also showed diminished expression of VEGFR2 in lung vascular ECs of gRNA3-transduced mice, as shown in FIG. 14C. VEGFR2 expression was also markedly inhibited in vascular ECs of heart (see FIG. 15) and aorta (see FIG. 16) of gRNA3-transduced mice.

The results of this example further demonstrate the highly efficient genome editing in ECs of the cardiovascular system by PP/PEI nanoparticle delivery of CRISPR^{CDH5} plasmid DNA in adult mice.

The above examples describe a simple and highly efficient approach to selectively target the cardiovascular endothelium through non-viral delivery of CRISPR plasmid DNA with the aid of nanoparticles. The PP/PEI nanoparticles described herein, coupled with human CDH5 promoter-driven expression of Cas9, induces EC-restricted genome editing in multiple organs including lung, heart, skeletal muscle vessels, and aorta of adult mice, thereby causing diminished protein expression in ECs to generate a phenotype similar to that seen in genetic knockout mice. Thus, the PP/PEI nanoparticles described herein provide one of the first systems for successful targeting of genome editing in organs other than the liver.

### Example 4

Although large molecular weight PEIs are potentially toxic due to their aggregation and difficulty degrading, low molecular weight PEIs (e.g. Mw-600, 1200 Da) are well tolerated. Low MW PEIs, however, exhibit low binding capacity for nucleic acids (Godbey et al., J Biomed Mater Res., 45: 268-275 (1999); Breunig et al., Proc Natl Acad Sci USA, 104: 14454-14459 (2007)). Modification of the large molecular weight PEI by succinylation has been shown to lower toxicity (Zintchenko et al., Bioconjug Chem., 19: 1448-55 (2008)). Cross-linking of low molecular weight PEI results in a DNA binding activity similar to PEI 25K Da.

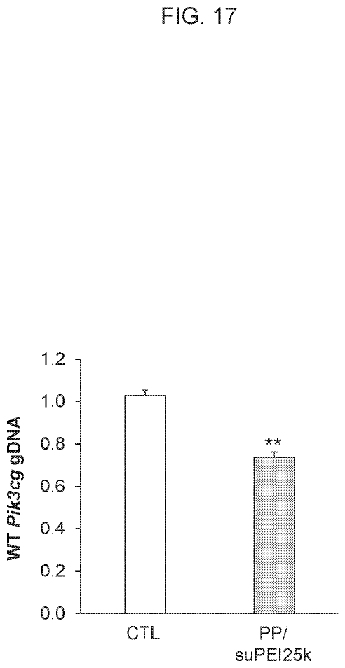

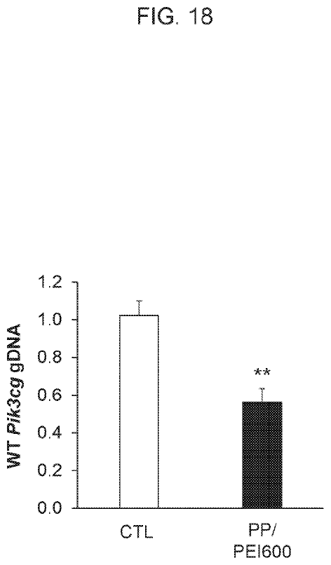

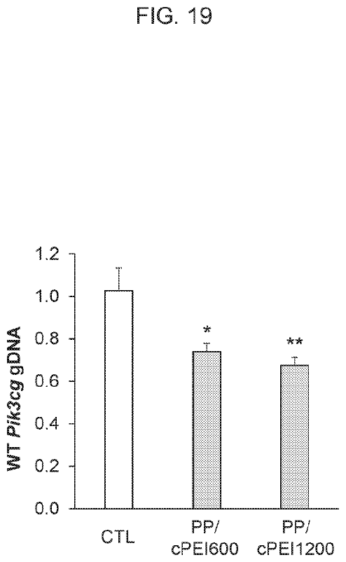

Succinylated PEI25k Da was formulated with the PP nanoparticle and mixed with CRISPR^{CDH5} plasmid DNA for delivery of Cas9/Pik3cg gRNA1 to adult mice. At 7 days post-administration, lung ECs were isolated for assessing genome editing efficacy, which was 30% (FIG. 17). PEI600 was formulated with PEG-PLGA nanoparticle in a specific ratio (e.g., 120:1) for delivery of Cas9/Pik3cg gRNA1 to adult mice. This formulation resulted in high efficiency in genome editing in lung ECs of Cas9/Pik3cg gRNA1 to adult mice (FIG. 18). This is the first demonstration that a novel formulation of low molecular weight PEI can efficiently deliver a nucleic acid in vivo and result in highly efficient genome editing which is similar to high molecular weight PEI. This new formulation is important as the resulting PP/PEI nanoparticle are potential safe for clinical use. Additionally, low molecular weight PEIs were also crosslinked by disulfide bonds to form a large molecular complex (cPEI) and then formulated with PLGA-PEG nanoparticle for in vivo gene delivery in adult mice. Marked genome editing was observed with these crosslinked low molecular weight PEIs (FIG. 19).

This example demonstrates highly efficient gene delivery and genome editing by novel formulation of low molecular weight PEI, or biodegradable crosslinked low molecular weight PEI, or modified large molecular weight PEI.

### Example 5

This example demonstrates highly efficient gene delivery in vivo using the nanoparticle composition described herein.

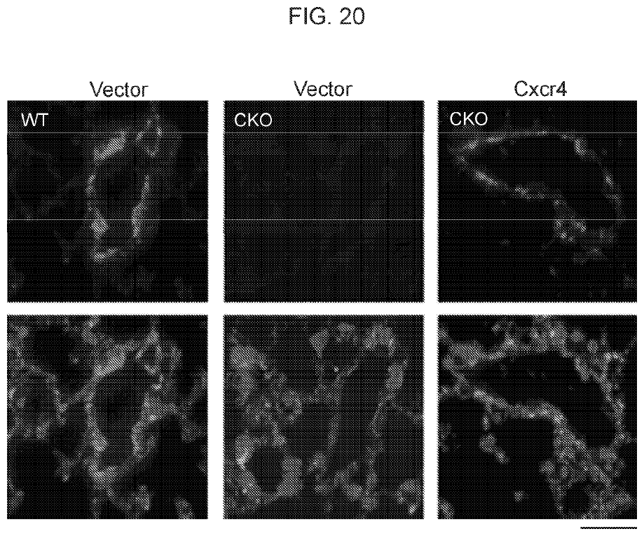

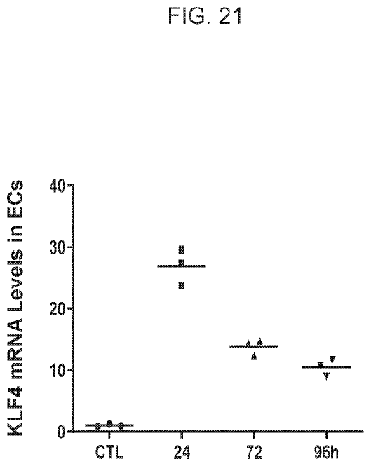

The PP/PEI nanoparticle was used for gene transfer and expression in vascular endothelial cells in vivo. At 12 hours post-lipopolysaccharide challenge, a mixture of PP/PEI:plasmid DNA expressing Cxcr4 under the control of a CDH5 promoter was administered to mice through retro-orbital injection. At 48 hours post-nanoparticle injection, Cxcr4 expression was restored in pulmonary vascular endothelial cells of Cxcr4 knockout mice administered with Cxcr4 plasmid DNA, as compared to empty vector (see FIG. 20). The kinetics of PP/PEI-mediated gene delivery also was assessed. A mixture of PP/PEI:plasmid DNA expressing Klf4 under the control of a CDH5 promoter was administered to adult mice under basal conditions through retro-orbital injection. PP/PEI:empty plasmid DNA (i.e., no Klf4 gene) was administrated as a control (CTL). Quantitative RT-PCR analysis demonstrated that PP/PEI:Klf4 plasmid DNA-treated mice expressed a 30-fold increase of KLF4 at 24 hour. The increased KLF4 expression lasted for at least 96 hours (FIG. 21).

### REFERENCES

The following references, as well as any references cited above, as herein incorporated by reference in their entireties.
1. Cines, D. B. et al. Endothelial cells in physiology and in the pathophysiology of vascular disorders. Blood 91, 3527-3561 (1998).
2. Mehta, D. & Malik, A. B. Signaling mechanisms regulating endothelial permeability. Physiol Rev 86, 279-367 (2006).
3. Munoz-Chapuli, R., Carmona, R., Guadix, J. A., Macias, D. & Perez-Pomares, J. M. The origin of the endothelial cells: an evo-devo approach for the invertebrate/vertebrate transition of the circulatory system. Evol Dev 7, 351-358 (2005).
4. Monahan-Earley, R., Dvorak, A. M. & Aird, W. C. Evolutionary origins of the blood vascular system and endothelium. J Thromb Haemost 11 Suppl 1, 46-66 (2013).
5. Ware, L. B. & Matthay, M. A. The acute respiratory distress syndrome. N Engl J Med 342, 1334-1349 (2000).
6. Cong, L. et al. Multiplex genome engineering using CRISPR/Cas systems. Science 339, 819-823 (2013).
7. Cox, D. B., Platt, R. J. & Zhang, F. Therapeutic genome editing: prospects and challenges. Nat Med 21, 121-131 (2015).
8. Doudna, J. A. & Charpentier, E. Genome editing. The new frontier of genome engineering with CRISPR-Cas9. Science 346, 1258096 (2014).
9. Yin, H. et al. Structure-guided chemical modification of guide RNA enables potent non-viral in vivo genome editing. Nat Biotechnol 35, 1179-1187 (2017).
10. Nelson, C. E. et al. In vivo genome editing improves muscle function in a mouse model of Duchenne muscular dystrophy. Science 351, 403-407 (2016).
11. Mali, P. et al. RNA-guided human genome engineering via Cas9. Science 339, 823-826 (2013).
12. Carroll, K. J. et al. A mouse model for adult cardiac-specific gene deletion with CRISPR/Cas9. Proc Natl Acad Sci USA 113, 338-343 (2016).
13. Tabebordbar, M. et al. In vivo gene editing in dystrophic mouse muscle and muscle stem cells. Science 351, 407-411 (2016).
14. Ran, F. A. et al. Genome engineering using the CRISPR-Cas9 system. Nat Protoc 8, 2281-2308 (2013).
15. Shalem, O., Sanjana, N. E. & Zhang, F. High-throughput functional genomics using CRISPR-Cas9. Nat Rev Genet 16, 299-311 (2015).
16. Wang, H. X. et al. CRISPR/Cas9-Based Genome Editing for Disease Modeling and Therapy: Challenges and Opportunities for Nonviral Delivery. Chem Rev 117, 9874-9906 (2017).
17. Gory, S. et al. The vascular endothelial-cadherin promoter directs endothelial specific expression in transgenic mice. Blood 93, 184-192 (1999).
18. Huang, X. et al. Endothelial p110gammaPI3K Mediates Endothelial Regeneration and Vascular Repair After Inflammatory Vascular Injury. Circulation 133, 1093-1103 (2016).
19. Prandini, M. H. et al. The human VE-cadherin promoter is subjected to organ specific regulation and is activated in tumour angiogenesis. Oncogene 24, 2992-3001 (2005).
20. Mirza, M. K. et al. FoxM1 regulates re-annealing of endothelial adherens junctions through transcriptional control of beta-catenin expression. J Exp Med 207, 1675-1685 (2010).
21. Zhao, Y. Y. et al. Endothelial cell-restricted disruption of FoxM1 impairs endothelial repair following LPS-induced vascular injury. J Clin Invest 116, 2333-2343 (2006).
22. Ertl, H. C. J. Preclinical models to assess the immunogenicity of AAV vectors. Cell Immunol (2017).
23. Staahl, B. T. et al. Efficient genome editing in the mouse brain by local delivery of engineered Cas9 ribonucleoprotein complexes. Nat Biotechnol 35, 431-434 (2017).
24. Wu, Z., Yang, H. & Colosi, P. Effect of genome size on AAV vector packaging. Mol Ther 18, 80-86 (2010).
25. Yuen, G. et al. CRISPR/Cas9-mediated gene knockout is insensitive to target copy number but is dependent on guide RNA potency and Cas9/sgRNA threshold expression level. Nucleic Acids Res 45, 12039-12053 (2017).
26. Zuris, J. A. et al. Cationic lipid-mediated delivery of proteins enables efficient protein-based genome editing in vitro and in vivo. Nat Biotechnol 33, 73-80 (2015).
27. Yin, H. et al. Therapeutic genome editing by combined viral and non-viral delivery of CRISPR system components in vivo. Nat Biotechnol 34, 328-333 (2016).
28. Zetsche, B. et al. Cpf1 is a single RNA-guided endonuclease of a class 2 CRISPR-Cas system. Cell 163, 759-771 (2015).
29. Abudayyeh, O. O. et al. RNA targeting with CRISPR-Cas13. Nature 550, 280-284 (2017).
30. Chen, J. S. et al. Enhanced proofreading governs CRISPR-Cas9 targeting accuracy. Nature 550, 407-410 (2017).
31. Kleinstiver, B. P. et al. High-fidelity CRISPR-Cas9 nucleases with no detectable genome-wide off-target effects. Nature 529, 490-495 (2016).

1. A composition comprising:
(a) a nanoparticle comprising a poly(Lactic Acid-co-Glycolic Acid) (PLGA)-b-polyethylene glycol (PEG)(PLGA-PEG) copolymer formulated with polyethylenimine (PEI), and
(b) one or more cargo molecules associated with the nanoparticle.

2. The composition of claim 1, wherein the one or more cargo molecules are nucleic acid molecules.

3. The composition of claim 2, wherein the one or more nucleic acid molecules is/are DNA and/or RNA.

4. The composition of claim 2, wherein the one or more nucleic acid molecules is plasmid DNA.

5. The composition of claim 2 or, wherein the one or more nucleic acid molecules is minicircle DNA.

6. The composition of claim 4, wherein the plasmid DNA or minicircle DNA expresses a gene, genome editor component(s), CRISPR/Cas9 components, or Cxrc4.

7. The composition of claim 2, wherein the one or more nucleic acid molecules is a small interfering RNA (siRNA), miRNA, long non-coding RNA (lncRNA), antisense RNA, coding RNA.

8. The composition of claim 1, wherein the one or more cargo molecules are small molecules.

9. The composition of claim 1, wherein the PEI is succinylated or unmodified large molecular weight PEI.

10. The composition of claim 1, wherein the ratio of cargo molecule to PLGA-PEG to PEI is about 1 μg:0.01-5 μg:0.1-100 μg.

11. The composition of claim 1, wherein the PEI is PEI25k.

12. The composition of claim 11, wherein the ratio of cargo molecule to PLGA-PEG to PEI is 1 μg plasmid DNA:0.375 μg:1.5 μg PEI25k.

13. The composition of claim 1, wherein the PEI is modified or unmodified low molecular weight PEI.

14. The composition of claim 13, wherein the modification is acetylation.

15. The composition of claim 1, wherein the PEI is acetylated or unmodified crosslinked low molecular weight PEI.

16. The composition of claim 15, wherein the crosslinker is disulfide, disimine, diacrylate.

17. The composition of claim 13, wherein the PEI is PEI 400, 600, 800, 1200, 1800.

18. The composition of claim 13, wherein the ratio of cargo molecule to PLGA-PEG to PEI is 1 μg plasmid DNA:0.375 μg:45 μg PEI600.

19. A method of delivering one or more cargo molecules to a cell, which comprises contacting the cell with the composition of claim 1, whereby the cargo molecule is delivered to the cell.

20. The method of claim 19, wherein the one or more cargo molecules are nucleic acid molecules.

21. The method of claim 19, wherein the cell is in vitro or in vivo.

22. The method of claim 19, wherein the cell is an endothelial cell or a fibroblast, a neuron cell, a hepatocyte, a cardiomyocyte, or a cancer cell.

23. The method of claim 19, wherein the nucleic acid molecule comprises a nucleic acid sequence that is operatively linked to a ubiquitous promoter or a cell- or tissue-specific promoter.

24. The method of claim 23, wherein the ubiquitous promoter is a CAG promoter.

25. The method of claim 23, wherein the nucleic acid molecule comprises a nucleic acid sequence that is operatively linked to an endothelial cell-specific promoter.

26. The method of claim 25, wherein the endothelial cell-specific promoter is a CDH5 promoter, a Tie2 promoter, or the 5′ endothelial enhancer of the stem cell leukemia locus.


SEQUENCE LISTING (54 sequences)

SEQ ID NO: 1 - DNA, 20 bp, Artificial sequence
  Synthetic
accgtaccacgacagtgcgc

SEQ ID NO: 2 - DNA, 20 bp, Artificial sequence
  Synthetic
atctggccagcgcactgtcg

SEQ ID NO: 3 - DNA, 20 bp, Artificial sequence
  Synthetic
agcctcgcaggtacgcctcc

SEQ ID NO: 4 - DNA, 20 bp, Artificial sequence
  Synthetic
actaaaagccggtaccctgg

SEQ ID NO: 5 - DNA, 21 bp, Artificial sequence
  Synthetic
ttgaaccgtaccacgacagtg

SEQ ID NO: 6 - DNA, 21 bp, Artificial sequence
  Synthetic
ctccaggatcacagcgaacct

SEQ ID NO: 7 - DNA, 21 bp, Artificial sequence
  Synthetic
ttaaccatgcagctcctggac

SEQ ID NO: 8 - DNA, 19 bp, Artificial sequence
  Synthetic
gaatctggccagcgcactg

SEQ ID NO: 9 - DNA, 21 bp, Artificial sequence
  Synthetic
ttgaaccgtaccacgacagtg

SEQ ID NO: 10 - DNA, 17 bp, Artificial sequence
  Synthetic
agcctcgcaggtacgcc

SEQ ID NO: 11 - DNA, 20 bp, Artificial sequence
  Synthetic
tggactaaaagccggtaccc

SEQ ID NO: 12 - DNA, 20 bp, Artificial sequence
  Synthetic
tcgttggataggactgtggg

SEQ ID NO: 13 - DNA, 21 bp, Artificial sequence
  Synthetic
ttgaaccgtaccacgacagtg

SEQ ID NO: 14 - DNA, 22 bp, Artificial sequence
  Synthetic
accagaacaagaagtgaccgat

SEQ ID NO: 15 - DNA, 20 bp, Artificial sequence
  Synthetic
gaactgtgggtttcccccat

SEQ ID NO: 16 - DNA, 19 bp, Artificial sequence
  Synthetic
gaatctggccagcgcactg

SEQ ID NO: 17 - DNA, 21 bp, Artificial sequence
  Synthetic
ttgaaccgtaccacgacagtg

SEQ ID NO: 18 - DNA, 17 bp, Artificial sequence
  Synthetic
agcctcgcaggtacgcc

SEQ ID NO: 19 - DNA, 20 bp, Artificial sequence
  Synthetic
tggactaaaagccggtaccc

SEQ ID NO: 20 - DNA, 21 bp, Artificial sequence
  Synthetic
tggtgctagtgatgagagggt

SEQ ID NO: 21 - DNA, 20 bp, Artificial sequence
  Synthetic
tgctaagcaagtgcctcctc

SEQ ID NO: 22 - DNA, 20 bp, Artificial sequence
  Synthetic
cttctctgccgagagcgatt

SEQ ID NO: 23 - DNA, 20 bp, Artificial sequence
  Synthetic
tggaatggttagatccccaa

SEQ ID NO: 24 - DNA, 21 bp, Artificial sequence
  Synthetic
tcagggaacttcacctatgga

SEQ ID NO: 25 - DNA, 20 bp, Artificial sequence
  Synthetic
ggcggtggtgacagtatctt

SEQ ID NO: 26 - DNA, 18 bp, Artificial sequence
  Synthetic
cgtcccggtacgagcact

SEQ ID NO: 27 - DNA, 20 bp, Artificial sequence
  Synthetic
ggcggtggtgacagtatctt

SEQ ID NO: 28 - DNA, 20 bp, Artificial sequence
  Synthetic
cggtgatgtacacgatgcca

SEQ ID NO: 29 - DNA, 25 bp, Artificial sequence
  Synthetic
ggcaaatacaacccttcagattact

SEQ ID NO: 30 - DNA, 18 bp, Artificial sequence
  Synthetic
cgtcccggtacgagcact

SEQ ID NO: 31 - DNA, 21 bp, Artificial sequence
  Synthetic
actggagcctacaagtgctcg

SEQ ID NO: 32 - DNA, 20 bp, Artificial sequence
  Synthetic
cggtgatgtacacgatgcca

SEQ ID NO: 33 - DNA, 20 bp, Artificial sequence
  Synthetic
aaactgtggtgatcccctgc

SEQ ID NO: 34 - DNA, 23 bp, Artificial sequence
  Synthetic
gagagtaaagcctatctcgctgt

SEQ ID NO: 35 - DNA, 20 bp, Artificial sequence
  Synthetic
agaatttcctgggacagcga

SEQ ID NO: 36 - DNA, 21 bp, Artificial sequence
  Synthetic
ttgcctcacagaagaccatgc

SEQ ID NO: 37 - DNA, 20 bp, Artificial sequence
  Synthetic
ggcggtggtgacagtatctt

SEQ ID NO: 38 - DNA, 18 bp, Artificial sequence
  Synthetic
cgtcccggtacgagcact

SEQ ID NO: 39 - DNA, 21 bp, Artificial sequence
  Synthetic
gatgtccgcattcatgcaagt

SEQ ID NO: 40 - DNA, 20 bp, Artificial sequence
  Synthetic
cggtgatgtacacgatgcca

SEQ ID NO: 41 - DNA, 23 bp, Artificial sequence
  Synthetic
agtggaaattgttgtgacctcag

SEQ ID NO: 42 - DNA, 23 bp, Artificial sequence
  Synthetic
agtggaaattgttgtgacctcag

SEQ ID NO: 43 - DNA, 21 bp, Artificial sequence
  Synthetic
actggagcctacaagtgctcg

SEQ ID NO: 44 - DNA, 20 bp, Artificial sequence
  Synthetic
acgacattggaagcagacgg

SEQ ID NO: 45 - DNA, 23 bp, Artificial sequence
  Synthetic
cgttgaatttagacagtgctggg

SEQ ID NO: 46 - DNA, 23 bp, Artificial sequence
  Synthetic
gagagtaaagcctatctcgctgt

SEQ ID NO: 47 - DNA, 20 bp, Artificial sequence
  Synthetic
agaatttcctgggacagcga

SEQ ID NO: 48 - DNA, 21 bp, Artificial sequence
  Synthetic
ttgcctcacagaagaccatgc

SEQ ID NO: 49 - DNA, 21 bp, Artificial sequence
  Synthetic
gagcgtgatatccttggtccc

SEQ ID NO: 50 - DNA, 20 bp, Artificial sequence
  Synthetic
gtctccatagctcctgtcgg

SEQ ID NO: 51 - DNA, 20 bp, Artificial sequence
  Synthetic
gggcaaagtcaatcccacct

SEQ ID NO: 52 - DNA, 20 bp, Artificial sequence
  Synthetic
agcccagaacatcaagccag

SEQ ID NO: 53 - DNA, 20 bp, Artificial sequence
  Synthetic
atgtgcagactagcctgtgg

SEQ ID NO: 54 - DNA, 20 bp, Artificial sequence
  Synthetic
agaggagaaaccccagttgc